(19) United States
(12) Patent Application Publication
(10) Pub. No.: US 2023/0234941 A1
(43) Pub. Date: Jul. 27, 2023

(54) Salts of Compound and Pharmaceutical Compositions Containing the Same

(71) Applicant: Shenzhen Forward Pharmaceuticals Co., Limited., Shenzhen (CN)

(72) Inventors: Chenggang Zhu, Shenzhen (CN); Xuan Yang, Shenzhen (CN); Chaochun Zhang, Shenzhen (CN); Liangliang Xu, Shenzhen (CN)

(21) Appl. No.: 18/002,578

(22) PCT Filed: Jun. 23, 2021
(86) PCT No.: PCT/CN2021/101778

§ 371 Date: Dec. 20, 2022

(30) Foreign Application Priority Data
Jun. 23, 2020 (CN) 202010579560.0
May 22, 2021 (CN) 202110563762.2

(51) Int. Cl. C07D 403/04

(52) U.S. Cl. CPC C07D 403/04

(57) ABSTRACT

The present application relates to a salt formed from a compound of formula (I) with an acid:

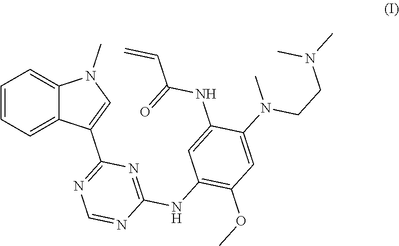 [SMILES: C=CC(=O)Nc1cc(Nc2ncnc(-c3cn(C)c4ccccc34)n2)c(OC)cc1N(C)CCN(C)C]

wherein the acid is selected from hydrochloric acid, methanesulfonic acid, benzenesulfonic acid, ethanesulfonic acid, maleic acid, hydrobromic acid, citric acid, L-tartaric acid, and p-toluenesulfonic acid. The present application also relates to a method for treating non-small cell lung cancer (NSCLC) with EGFR exon 20 insertion mutation using the salt.

### TECHNICAL FIELD

The present application relates to a salt formed from a compound of formula (I) with an acid:

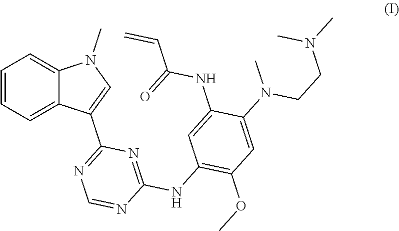 [SMILES: C=CC(=O)Nc1cc(Nc2ncnc(-c3cn(C)c4ccccc34)n2)c(OC)cc1N(C)CCN(C)C]

wherein the acid is selected from hydrochloric acid, methanesulfonic acid, benzenesulfonic acid, ethanesulfonic acid, maleic acid, hydrobromic acid, citric acid, L-tartaric acid, and p-toluenesulfonic acid. The present application also relates to a method for treating non-small cell lung cancer (NSCLC) with EGFR exon 20 insertion mutation using the salt.

### BACKGROUND ART

Chinese patent CN105461695B discloses a compound of formula (I), which have multiple basic centers. The inhibitory activity of the compound against EGFR activating mutation (such as exon 19 deletion activating mutation, L858R activating mutation, T790M drug resistance mutation and exon 20 insertion mutation) is significantly higher than the inhibitory activity against wild-type EGFR (WT EGFR), and therefore, the compound has higher selectivity and safety, and lower toxic and side effects.

Salt formation studies are usually carried out on organic basic compounds having activity. However, those skilled in the art cannot predict with which acids a specific organic basic compound can form stable salts, or whether a specific organic basic compound or its acid addition salt is more suitable for further drug development, let alone which salt formed has better chemical stability, physical stability or solubility, and which salt has all these better properties. In particular, if an organic basic compound has multiple basic centers, it is impossible for those skilled in the art to predict whether its salts formed with a specific acid in various equivalent ratios have the same properties or different properties, let alone which equivalent ratio of the organic basic compound to the acid is more suitable for further drug development.

### SUMMARY OF THE INVENTION

The present application seeks to find salts of the compound of formula (I) suitable for further drug development. In particular, the present application seeks to find salts with properties suitable for further drug development, including a reasonable salt-forming equivalent ratio, better chemical stability, better physical stability and/or better solubility.

In one aspect according to the invention, the present application relates to a salt formed from the compound of formula (I) with an acid:

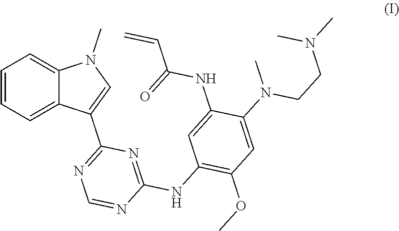 [SMILES: C=CC(=O)Nc1cc(Nc2ncnc(-c3cn(C)c4ccccc34)n2)c(OC)cc1N(C)CCN(C)C]

wherein the acid is selected from hydrochloric acid, methanesulfonic acid, benzenesulfonic acid, ethanesulfonic acid, maleic acid, hydrobromic acid, citric acid, L-tartaric acid, and p-toluenesulfonic acid.

In another aspect according to the invention, the present application relates to a pharmaceutical composition comprising the salt according to the invention, which comprises the salt according to the invention and a pharmaceutically acceptable carrier.

In still another aspect according to the invention, the present application relates to a method of treating non-small cell lung cancer (NSCLC) with EGFR exon 20 insertion mutation, comprising administering the salt according to the invention to a patient.

In a further aspect according to the invention, the present application relates to the use of the salt according to the invention in the preparation of a medicament for the treatment of non-small cell lung cancer (NSCLC) with EGFR exon 20 insertion mutation.

### BRIEF DESCRIPTION OF THE DRAWINGS

FIG. 1: XRPD pattern of the amorphous free base (Sample No. Y11526-45-RV-FWD1509-AF-SU12).

FIG. 2: DSC profile of the amorphous free base (Sample No. Y11526-45-RV-FWD1509-AF-SU12).

FIG. 3: TGA profile of the amorphous free base (Sample No. Y11526-45-RV-FWD1509-AF-SU12).

FIG. 4: ¹H-NMR spectrum of the amorphous free base (Sample No. Y11526-45-RV-FWD1509-AF-SU12).

FIG. 5: PLM photograph of the amorphous free base (Sample No. Y11526-45-RV-FWD1509-AF-SU12).

FIG. 6: XRPD pattern of the amorphous 1 eq. (equivalent) hydrochloride salt (Sample No. Y11526-42-SU11-methanol-dichloromethane).

FIG. 7: DSC profile of the amorphous 1 eq. hydrochloride salt (Sample No. Y11526-42-SU11-methanol-dichloromethane).

FIG. 8: TGA profile of the amorphous 1 eq. hydrochloride salt (Sample No. Y11526-42-SU11-methanol-dichloromethane).

FIG. 9: ¹H-NMR spectrum of the amorphous 1 eq. hydrochloride salt (Sample No. Y11526-42-SU11-methanol-dichloromethane).

FIG. 10: PLM photograph of the amorphous 1 eq. hydrochloride salt (Sample No. Y11526-42-SU11-methanol-dichloromethane).

FIG. 11: XRPD pattern of the amorphous 2 eq. hydrochloride salt (Sample No. Y11526-28-SU5-methanol-dichloromethane).

FIG. 12: DSC profile of the amorphous 2 eq. hydrochloride salt (Sample No. Y11526-28-SU5-methanol-dichloromethane).

FIG. 13: TGA profile of the amorphous 2 eq. hydrochloride salt (Sample No. Y11526-28-SU5-methanol-dichloromethane).

FIG. 14: ¹H-NMR spectrum of the amorphous 2 eq. hydrochloride salt (Sample No. Y11526-28-SU5-methanol-dichloromethane).

FIG. 15: PLM photograph of the amorphous 2 eq. hydrochloride salt (Sample No. Y11526-28-SU5-methanol-dichloromethane).

FIG. 16: XRPD pattern of the amorphous 1 eq. mesylate salt (Sample No. Y11526-42-SU10-methanol-dichloromethane).

FIG. 17: DSC profile of the amorphous 1 eq. mesylate salt (Sample No. Y11526-42-SU10-methanol-dichloromethane).

FIG. 18: TGA profile of the amorphous 1 eq. mesylate salt (Sample No. Y11526-42-SU10-methanol-dichloromethane).

FIG. 19: ¹H-NMR spectrum of the amorphous 1 eq. mesylate salt (Sample No. Y11526-42-SU10-methanol-dichloromethane).

FIG. 20: PLM photograph of the amorphous 1 eq. mesylate salt (Sample No. Y11526-42-SU10-methanol-dichloromethane).

FIG. 21: XRPD pattern of the amorphous 2 eq. mesylate salt (Sample No. Y11526-28-SU4-methanol-dichloromethane).

FIG. 22: DSC profile of the amorphous 2 eq. mesylate salt (Sample No. Y11526-28-SU4-methanol-dichloromethane).

FIG. 23: TGA profile of the amorphous 2 eq. mesylate salt (Sample No. Y11526-28-SU4-methanol-dichloromethane).

FIG. 24: ¹H-NMR spectrum of the amorphous 2 eq. mesylate salt (Sample No. Y11526-28-SU4-methanol-dichloromethane).

FIG. 25: PLM photograph of the amorphous 2 eq. mesylate salt (Sample No. Y11526-28-SU4-methanol-dichloromethane).

FIG. 26: XRPD pattern of the amorphous 1 eq. besylate salt (Sample No. Y11526-42-SU9-methanol-dichloromethane).

FIG. 27: DSC profile of the amorphous 1 eq. besylate salt (Sample No. Y11526-42-SU9-methanol-dichloromethane).

FIG. 28: TGA profile of the amorphous 1 eq. besylate salt (Sample No. Y11526-42-SU9-methanol-dichloromethane).

FIG. 29: ¹H-NMR spectrum of the amorphous 1 eq. besylate salt (Sample No. Y11526-42-SU9-methanol-dichloromethane).

FIG. 30: PLM photograph of the amorphous 1 eq. besylate salt (Sample No. Y11526-42-SU9-methanol-dichloromethane).

FIG. 31: XRPD pattern of the amorphous 2 eq. besylate salt (Sample No. Y11526-30-SU1-water).

FIG. 32: DSC profile of the amorphous 2 eq. besylate salt (Sample No. Y11526-30-SU1-water).

FIG. 33: TGA profile of the amorphous 2 eq. besylate salt (Sample No. Y11526-30-SU1-water).

FIG. 34: ¹H-NMR spectrum of the amorphous 2 eq. besylate salt (Sample No. Y11526-30-SU1-water).

FIG. 35: PLM photograph of the amorphous 2 eq. besylate salt (Sample No. Y11526-30-SU1-water).

FIG. 36: XRPD pattern of the amorphous 1 eq. esylate salt (Sample No. Y11526-28-SU7-methanol-dichloromethane).

FIG. 37: DSC profile of the amorphous 1 eq. esylate salt (Sample No. Y11526-28-SU7-methanol-dichloromethane).

FIG. 38: TGA profile of the amorphous 1 eq. esylate salt (Sample No. Y11526-28-SU7-methanol-dichloromethane).

FIG. 39: ¹H-NMR spectrum of the amorphous 1 eq. esylate salt (Sample No. Y11526-28-SU7-methanol-dichloromethane).

FIG. 40: PLM photograph of the amorphous 1 eq. esylate salt (Sample No. Y11526-28-SU7-methanol-dichloromethane).

FIG. 41: XRPD pattern of the amorphous 1 eq. maleate salt (Sample No. Y11526-30-SU2-water).

FIG. 42: DSC profile of the amorphous 1 eq. maleate salt (Sample No. Y11526-30-SU2-water).

FIG. 43: TGA profile of the amorphous 1 eq. maleate salt (Sample No. Y11526-30-SU2-water).

FIG. 44: ¹H-NMR spectrum of the amorphous 1 eq. maleate salt (Sample No. Y11526-30-SU2-water).

FIG. 45: PLM photograph of the amorphous 1 eq. maleate salt (Sample No. Y11526-30-SU2-water).

FIG. 46: XRPD pattern of the crystalline 1 eq. hydrobromide salt (Sample No. Y11526-33-SU8-methanol-dichloromethane).

FIG. 47: DSC profile of the crystalline 1 eq. hydrobromide salt (Sample No. Y11526-33-SU8-methanol-dichloromethane).

FIG. 48: TGA profile of the crystalline 1 eq. hydrobromide salt (Sample No. Y11526-33-SU8-methanol-dichloromethane).

FIG. 49: ¹H-NMR spectrum of the crystalline 1 eq. hydrobromide salt (Sample No. Y11526-33-SU8-methanol-dichloromethane).

FIG. 50: PLM photograph of the crystalline 1 eq. hydrobromide salt (Sample No. Y11526-33-SU8-methanol-dichloromethane).

FIG. 51: XRPD pattern of the amorphous nitrate salt (Sample No. Y11526-18-RV7-methanol-dichloromethane).

FIG. 52: ¹H-NMR spectrum of the amorphous nitrate salt (Sample No. Y11526-18-RV7-methanol-dichloromethane).

FIG. 53: XRPD pattern of the amorphous sulfate salt (Sample No. Y11526-15-RV3-methanol-dichloromethane).

FIG. 54: DSC profile of the amorphous sulfate salt (Sample No. Y11526-15-RV3-methanol-acetonitrile).

FIG. 55: TGA profile of the amorphous sulfate salt (Sample No. Y11526-15-RV3-methanol-acetonitrile).

FIG. 56: ¹H-NMR spectrum of the amorphous sulfate salt (Sample No. Y11526-15-RV3-methanol-acetonitrile).

FIG. 57: PLM photograph of the amorphous sulfate salt (Sample No. Y11526-15-RV3-methanol-acetonitrile).

FIG. 58: XRPD pattern of the amorphous 2 eq. p-tosylate salt (Sample No. Y11526-23-FD11-water).

FIG. 59: DSC profile of the amorphous 2 eq. p-tosylate salt (Sample No. Y11526-23-FD11-water).

FIG. 60: TGA profile of the amorphous 2 eq. p-tosylate salt (Sample No. Y11526-23-FD11-water).

FIG. 61: ¹H-NMR spectrum of the amorphous 2 eq. p-tosylate salt (Sample No. Y11526-23-FD11-water).

FIG. 62: PLM photograph of the amorphous 2 eq. p-tosylate salt (Sample No. Y11526-23-FD11-water).

FIG. 63: photograph of the amorphous 2 eq. p-tosylate salt (Sample No. Y11526-23-FD11-water).

FIG. 64: XRPD pattern of the amorphous sulfosalicylate salt (Sample No. Y11526-25-FD13-1,4-dioxane).

FIG. 65: DSC profile of the amorphous sulfosalicylate salt (Sample No. Y11526-25-FD13-1,4-dioxane).

FIG. 66: TGA profile of the amorphous sulfosalicylate salt (Sample No. Y11526-25-FD13-1,4-dioxane).

FIG. 67: ¹H-NMR spectrum of the amorphous sulfosalicylate salt (Sample No. Y11526-25-FD13-1,4-dioxane).

FIG. 68: PLM photograph of the amorphous sulfosalicylate salt (Sample No. Y11526-25-FD13-1,4-dioxane).

FIG. 69: XRPD pattern of the amorphous sulfosalicylate salt (Sample No. Y11526-25-FD12-1,4-dioxane).

FIG. 70: DSC profile of the amorphous sulfosalicylate salt (Sample No. Y11526-25-FD12-1,4-dioxane).

FIG. 71: TGA profile of the amorphous sulfosalicylate salt (Sample No. Y11526-25-FD12-1,4-dioxane).

FIG. 72: ¹H-NMR spectrum of the amorphous sulfosalicylate salt (Sample No. Y11526-25-FD12-1,4-dioxane).

FIG. 73: PLM photograph of the amorphous sulfosalicylate salt (Sample No. Y11526-25-FD12-1,4-dioxane).

FIG. 74: XRPD pattern of the amorphous L-malate salt (Sample No. Y11526-17-RV6-methanol-dichloromethane).

FIG. 75: DSC profile of the amorphous L-malate salt (Sample No. Y11526-17-RV6-methanol-dichloromethane).

FIG. 76: TGA profile of the amorphous L-malate salt (Sample No. Y11526-17-RV6-methanol-dichloromethane).

FIG. 77: ¹H-NMR spectrum of the amorphous L-malate salt (Sample No. Y11526-17-RV6-methanol-dichloromethane).

FIG. 78: PLM photograph of the amorphous L-malate salt (Sample No. Y11526-17-RV6-methanol-dichloromethane).

FIG. 79: XRPD pattern of the amorphous 1 eq. citrate salt (Sample No. Y11526-10-FD6-1,4-dioxane).

FIG. 80: DSC profile of the amorphous 1 eq. citrate salt (Sample No. Y11526-10-FD6-1,4-dioxane).

FIG. 81: TGA profile of the amorphous 1 eq. citrate salt (Sample No. Y11526-10-FD6-1,4-dioxane).

FIG. 82: ¹H-NMR spectrum of the amorphous 1 eq. citrate salt (Sample No. Y11526-10-FD6-1,4-dioxane).

FIG. 83: PLM photograph of the amorphous 1 eq. citrate salt (Sample No. Y11526-10-FD6-1,4-dioxane).

FIG. 84: XRPD pattern of the amorphous 1 eq. L-tartrate salt (Sample No. Y11526-10-FD7-1,4-dioxane).

FIG. 85: DSC profile of the amorphous 1 eq. L-tartrate salt (Sample No. Y11526-10-FD7-1,4-dioxane).

FIG. 86: TGA profile of the amorphous 1 eq. L-tartrate salt (Sample No. Y11526-10-FD7-1,4-dioxane).

FIG. 87: ¹H-NMR spectrum of the amorphous 1 eq. L-tartrate salt (Sample No. Y11526-10-FD7-1,4-dioxane).

FIG. 88: HPLC chromatogram overlay of the amorphous 1 eq. L-tartrate salt (Sample No. Y11526-10-FD7-1,4-dioxane).

FIG. 89: XRPD overlay of the amorphous free base in a solid storage stability test, in which from top to bottom, the first trace is the XRPD of the crystalline free base as a control, the second trace is the solid obtained in Test BS2, the third trace is the XRPD of the solid obtained in Test BS1, and the fourth trace is the XRPD of the original solid.

FIG. 90: XRPD overlay of the amorphous 1 eq. hydrochloride salt in a solid storage stability test, in which the upper trace is the XRPD of the solid obtained in Test BS2, the middle trace is the XRPD of the solid obtained in Test BS1, and the lower trace is the XRPD of the original solid.

FIG. 91: XRPD overlay of the amorphous 2 eq. hydrochloride salt in a solid storage stability test, in which the upper trace is the XRPD of the solid obtained in Test BS2, the middle trace is the XRPD of the solid obtained in Test BS1, and the lower trace is the XRPD of the original solid.

FIG. 92: Photograph of the amorphous 2 eq. hydrochloride salt in a solid storage stability test, in which the left picture is the photograph of the original solid, and the right picture is the photograph of the solid obtained in Test BS1.

FIG. 93: XRPD overlay of the amorphous 1 eq. mesylate salt in a solid storage stability test, in which the upper trace is the XRPD of the solid obtained in Test BS2, the middle trace is the XRPD of the solid obtained in Test BS1, and the lower trace is the XRPD of the original solid.

FIG. 94: XRPD overlay of the amorphous 2 eq. mesylate salt in a solid storage stability test, in which the upper trace is the XRPD of the solid obtained in Test BS2, the middle trace is the XRPD of the solid obtained in Test BS1, and the lower trace is the XRPD of the original solid.

FIG. 95: Photograph of the amorphous 2 eq. mesylate salt in a solid storage stability test, in which the left picture is the photograph of the original solid, the middle picture is the photograph of the solid obtained in Test BS1, and the right picture is the photograph of the solid obtained in Test BS2.

FIG. 96: XRPD overlay of the amorphous 1 eq. besylate salt in a solid storage stability test, in which the upper trace is the XRPD of the solid obtained in Test BS2, the middle trace is the XRPD of the solid obtained in Test BS 1, and the lower trace is the XRPD of the original solid.

FIG. 97: Photograph of the amorphous 2 eq. besylate salt in a solid storage stability test, in which the left picture is the photograph of the original solid, and the right picture is the photograph of the solid obtained in Test BS1.

FIG. 98: XRPD overlay of the amorphous 2 eq. besylate salt in a solid storage stability test, in which the upper trace is the XRPD of the solid obtained in Test BS2, and the lower trace is the XRPD of the original solid.

FIG. 99: Photograph of the amorphous 1 eq. esylate salt in a solid storage stability test, in which the left picture is the photograph of the original solid, and the right picture is the photograph of the solid obtained in Test BS1.

FIG. 100: XRPD overlay of the amorphous 1 eq. esylate salt in a solid storage stability test, in which the upper trace is the XRPD of the solid obtained in Test BS2, and the lower trace is the XRPD of the original solid.

FIG. 101: XRPD overlay of the amorphous 1 eq. maleate salt in a solid storage stability test, in which the upper trace is the XRPD of the solid obtained in Test BS2, the middle trace is the XRPD of the solid obtained in Test BS1, and the lower trace is the XRPD of the original solid.

FIG. 102: Photograph of the amorphous 1 eq. maleate salt in a solid storage stability test, in which the left picture is the photograph of the original solid, and the right picture is the photograph of the solid obtained in Test BS1.

FIG. 103: XRPD overlay of the crystalline 1 eq. hydrobromide salt in a solid storage stability test, in which the upper trace is the XRPD of the solid obtained in Test BS2, the middle trace is the XRPD of the solid obtained in Test BS1, and the lower trace is the XRPD of the original solid.

FIG. 104: XRPD pattern of the solid of the crystalline 1 eq. hydrobromide salt obtained in a 2 h solid solubility test, in which the upper trace is the XRPD of the solid obtained in the 2 h solid solubility test, and the lower trace is the XRPD of the original solid.

### DETAILED DESCRIPTION OF THE INVENTION

In one aspect, the present application relates to a salt formed from a compound of formula (I) with an acid:

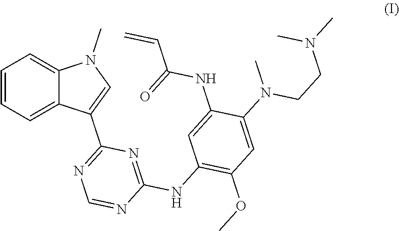 [SMILES: C=CC(=O)Nc1cc(Nc2ncnc(-c3cn(C)c4ccccc34)n2)c(OC)cc1N(C)CCN(C)C]

wherein the acid is selected from hydrochloric acid, methanesulfonic acid, benzenesulfonic acid, ethanesulfonic acid, maleic acid, hydrobromic acid, citric acid, L-tartaric acid, and p-toluenesulfonic acid. Preferably, the acid is selected from hydrochloric acid, methanesulfonic acid, and maleic acid. More preferably, the equivalent ratio of the compound of formula (I) to the acid is 1:1 or 1:2. Further preferably, the acid is methanesulfonic acid. More further preferably, the equivalent ratio of the compound of formula (I) to the acid is 1:1.

The inventors of the present application have found that when the compound of formula (I) is reacted with an organic or inorganic acid, it is unexpected that the compound of formula (I) can form salts with some acids, but cannot form salts with other acids at all.

More surprisingly, when the compound of formula (I) is reacted with an acid selected from hydrochloric acid, methanesulfonic acid, benzenesulfonic acid, ethanesulfonic acid, maleic acid, hydrobromic acid, sulfosalicylic acid, L-malic acid, citric acid, or L-tartaric acid in a molar charge ratio of compound of formula (I):acid of 1:1, some of the salts formed have a stoichiometric ratio of compound of formula (I):acid of 1:1, while the compound of formula (I) and some acids cannot form salts having a stoichiometric ratio of compound of formula (I):acid of 1:1. Specifically:

hydrochloric acid, methanesulfonic acid, benzenesulfonic acid, ethanesulfonic acid, maleic acid, hydrobromic acid, citric acid, and L-tartaric acid enable the resulting salts to have a stoichiometric ratio of compound of formula (I):acid of 1:1 (the corresponding salts are hereinafter referred to as 1 eq. hydrochloride salt, 1 eq. mesylate salt, 1 eq. besylate salt, 1 eq. esylate salt, 1 eq. maleate salt, 1 eq. hydrobromide salt, 1 eq. citrate salt and 1 eq. L-tartrate, respectively), while sulfosalicylic acid and L-malic acid cannot enable the resulting salts to have a stoichiometric ratio of compound of formula (I):acid of 1:1.

In addition, more surprisingly, when the compound of formula (I) is reacted with an acid selected from hydrochloric acid, methanesulfonic acid, benzenesulfonic acid, nitric acid, sulfuric acid, p-toluenesulfonic acid, or sulfosalicylic acid in a molar charge ratio of compound of formula (I):acid of 1:2, some of the salts formed have a stoichiometric ratio of compound of formula (I):acid of 1:2, while the compound of formula (I) and some acids cannot form salts having a stoichiometric ratio of compound of formula (I):acid of 1:2. Specifically:

hydrochloric acid, methanesulfonic acid, benzenesulfonic acid, and p-toluenesulfonic acid enable the resulting salts to have a stoichiometric ratio of compound of formula (I):acid of 1:2 (the corresponding salts are hereinafter referred to as 2 eq. hydrochloride salt, 2 eq. mesylate salt, 2 eq. besylate salt, and 2 eq. p-tosylate salt, respectively), while nitric acid, sulfuric acid, and sulfosalicylic acid cannot enable the resulting salts to have a stoichiometric ratio of compound of formula (I):acid of 1:2.

More specifically, the salts obtained as above include 1 eq. hydrochloride salt, 1 eq. mesylate salt, 1 eq. besylate salt, 1 eq. esylate salt, 1 eq. maleate salt, 1 eq. hydrobromide salt, 1 eq. citrate salt, 1 eq. L-tartrate, 2 eq. hydrochloride salt, 2 eq. mesylate salt, 2 eq. besylate salt and 2 eq. p-tosylate salt, and sulfosalicylate salt, L-malate salt, nitrate salt, and sulfate salt that do not have a reasonable salt-forming equivalent ratio. For example, the salts obtained as above include amorphous 1 eq. hydrochloride salt, amorphous 1 eq. mesylate salt, amorphous 1 eq. besylate salt, amorphous 1 eq. esylate salt, amorphous 1 eq. maleate salt, crystalline 1 eq. hydrobromide salt, amorphous 1 eq. citrate salt, amorphous 1 eq. L-tartrate, amorphous 2 eq. hydrochloride salt, amorphous 2 eq. mesylate salt, amorphous 2 eq. besylate salt and amorphous 2 eq. p-tosylate salt, and amorphous sulfosalicylate salt, amorphous L-malate salt, amorphous nitrate salt, and amorphous sulfate salt that do not have a reasonable salt-forming equivalent ratio.

It is crucial for drug development whether the salts obtained as above have chemical and physical stability at the end of the preparation. The chemical and physical stability of the salts at the end of the preparation includes:

the chemical stability of the salts at the end of the preparation, that is, the purity of the salts obtained at the end of the preparation is not significantly reduced compared with the purity of the free base used to prepare the salts; and

the physical stability of the salts at the end of the preparation, that is, the salts do not undergo crystal phase transformation, moisture absorption and/or color change, etc. immediately at the end of the preparation.

Different salts behave differently in terms of chemical and physical stability at the end of the preparation, indicating that it is unpredictable how a certain salt behaves. Specifically:

1 eq. citrate salt, 1 eq. L-tartrate, nitrate salt and L-malate salt are chemically unstable at the end of the preparation;

2 eq. p-tosylate salt and sulfate salt are physically unstable at the end of the preparation; and

1 eq. hydrochloride salt, 2 eq. hydrochloride salt, 1 eq. mesylate salt, 2 eq. mesylate salt, 1 eq. besylate salt, 2 eq. besylate salt, 1 eq. esylate salt, 1 eq. maleate salt, 1 eq. hydrobromide salt, and sulfosalicylate salt are chemically and physically stable at the end of the preparation.

The salts as above which have a reasonable salt-forming equivalent ratio and are chemically and physically stable at the end of the preparation include 1 eq. hydrochloride salt, 2 eq. hydrochloride salt, 1 eq. mesylate salt, 2 eq. mesylate salt, 1 eq. besylate salt, 2 eq. besylate salt, 1 eq. esylate salt, 1 eq. maleate salt, and 1 eq. hydrobromide salt. It is also crucial for drug development whether these salts remain chemically and physically stable after storage. The chemical and physical stability of the salts after storage includes:

the chemical stability of the salts after storage, that is, the purity of the salts after storage is not significantly reduced compared with the purity of the salts before storage; and

the physical stability of the salts after storage, that is, the salts do not undergo crystal phase transformation, moisture absorption and/or color change, etc. after storage.

Different salts behave differently in terms of chemical and physical stability after storage, indicating that it is unpredictable how a certain salt behaves.

Specifically:

1 eq. hydrochloride salt, 1 eq. besylate salt, and 1 eq. maleate salt are chemically unstable under storage conditions of “solid/25° C./60% RH/open/1 week” and/or “solid/60° C./closed container/1 week”;

2 eq. hydrochloride salt, 2 eq. mesylate salt, 2 eq. besylate salt, 1 eq. esylate salt, and 1 eq. maleate salt are physically unstable under storage conditions of “solid/25° C./60% RH/open/l week” and/or “solid/60° C./closed container/1 week”; and

1 eq. mesylate salt and 1 eq. hydrobromide salt are chemically and physically stable under storage conditions of “solid/25° C./60% RH/open/l week” and “solid/60° C./closed container/week”.

The salts as above which have a reasonable salt-forming equivalent ratio and are chemically and physically stable at the end of the preparation include 1 eq. hydrochloride salt, 2 eq. hydrochloride salt, 1 eq. mesylate salt, 2 eq. mesylate salt, 1 eq. besylate salt, 2 eq. besylate salt, 1 eq. esylate salt, 1 eq. maleate salt, and 1 eq. hydrobromide salt. It is also crucial for drug development whether these salts have better solubility. Different salts have different solubility, indicating that it is unpredictable what solubility a certain salt may have. Specifically:

2 eq. besylate salt and 1 eq. hydrobromide salt do not achieve a solubility of 2 mg/mL in some solvents that mimic physiological conditions; and

1 eq. hydrochloride salt, 2 eq. hydrochloride salt, 1 eq. mesylate salt, 2 eq. mesylate salt, 1 eq. besylate salt, 1 eq. esylate salt, and 1 eq. maleate salt have a solubility greater than 2 mg/mL in various solvents that mimic physiological conditions.

As can be seen from the above contents, 1 eq. mesylate salt surprisingly and unexpectedly has a reasonable salt-forming equivalent ratio, better chemical stability, better physical stability and better solubility simultaneously, which makes it as a salt of the compound of formula (I) suitable for further drug development.

In another aspect according to the invention, the present application relates to a pharmaceutical composition comprising the salt according to the invention, which comprises the salt according to the invention and a pharmaceutically acceptable carrier.

In still another aspect according to the invention, the present application relates to a method of treating non-small cell lung cancer (NSCLC) with EGFR exon 20 insertion mutation, comprising administering the salt according to the invention to a patient.

In a further aspect according to the invention, the present application relates to the use of the salt according to the invention in the preparation of a medicament for the treatment of non-small cell lung cancer (NSCLC) with EGFR exon 20 insertion mutation.

The present application will be illustrated by the following examples.

### EXAMPLES

The follow experimental conditions were used in the examples

X-ray powder diffractometer (XRPD)

| | |
|---|---|
| Instrument | Broker D8 Advance |
| Detector | LYNXEYE_XE_T(1D model) |
| Opening angle | 2.94° |
| Scanning mode | Continuous PSD fast mode |
| Radiation source | Cu/K-Alpha 1 (λ = 1.5418Å) |
| X-ray source power | 40 kV, 40 mA |
| Step size | 0.02° |
| Time per step | 0.06 seconds/step |
| Scanning range | 3° to 40° |
| Slit in primary | Twin_Secondary Motorized slit 10.0 mm |
| beam path | according to sample length; |
| | SollerMount axial Seller angle 2.5° |
| Slit in secondary | Detector OpticsMount Soller slit 2.5°; |
| beam path | Twin_Secondary Motorized slit 5.2 mm |
| Rotation speed | 15 rpm |
| of sample | |
| XRPD plate or | monocrystalline silicon wafer, flat plate |
| Instrument | Bruker D8 Advance |
| Detector | LYNXEYE_XE_T (1D model) |
| Opening angle | 2.94° |
| Scanning mode | Continuous PSD fast mode |
| Radiation source | Cu/K-Alpha 1 (λ = 1.5418Å) |
| X-ray source power | 40 kV, 40 mA |
| Step size | 0.02° |
| Time per step | 0.12 seconds/step |
| Scanning range | 3° to 40° |
| Slit in primary | Twin_Secondary Motorized slit 10.0 mm |
| beam path | according to sample length; |
| | SollerMount axial Seller angle 2.5° |
| Slit in secondary | Detector OpticsMount Seller slit 2.5°; |
| beam path | Twin_Secondary Motorized slit 5.2 mm |
| Rotation speed | 15 rpm |
| of sample | |
| XRPD plate | monocrystalline silicon wafer, flat plate |

Differential scanning calorimetry (DSC)

| | |
|---|---|
| Instrument | TA Discovery 2500 or Q2000 |
| Sample pan | Tzero pan and Tzero sealing cap with pinhole |
| Temperature range | 30 to 250° C. or before degradation |
| Rate of | 10° C./min or 2° C./min |
| temperature rise | |
| Flow rate of nitrogen | 50 mL/min |
| Sample amount | about 1 to 2 mg |

Thermogravimetic analysis (TGA)

| | |
|---|---|
| Instrument | Discovery 5500 or Q5000 |
| Sample pan | aluminum pan, open |
| Flow rate of nitrogen | 10 mL/min for balance; 25 mL/min for sample |
| Initial temperature | Environmental condition (lower than 35° C.) |
| Final temperature | 300° C. or abort next segment if |
| | weight <80% (w/w) (the weight loss of |
| | the compound is not greater than 20% (w/w)) |
| Rate of | 10° C./min |
| temperature rise | |
| Sample amount | about 2 to 10 mg |

Polarizing microscope (PLM)

| | |
|---|---|
| Instrument | Nikon LV100POL |
| Method | crossed polarizer, with the addition of silicone oil |

Magnetic resonance imaging (NMR)

| | |
|---|---|
| Instrument | Bruker Avance-AV 400M |
| Frequency | 400 MHz |
| Probe | 5 mm PABBO BB/19F-1H/D |
| | Z-GRD Z108618/0406 |
| Number of scans | 8 |
| Temperature | 297.6K |
| Relaxation | 1 second |

High performance liquid chromatography (HPLC)

| Instrument | Shimadzu LC40 | | |
|---|---|---|---|
| Wavelength | 210 nm | | |
| Column | Waters Xbridge C18 3.5 μm, 4.6*150 mm | | |
| Detector | DAD | | |
| Column | 40° C. | | |
| temperature | | | |
| Flow rate | 1.0 mL/min | | |
| Mobile phase A | 20 mmol/L dipotassium hydrogen phosphate solution | | |
| | (adjusted with phosphoric acid to pH = 8.05) | | |
| Mobile phase B | acetonitrile | | |
| Diluent | acetonitrile/water (v:v = 1:1) | | |
| Injection volume | 5 μL | | |
| | | Mobile | Mobile |
| | Time | phase | phase |
| | (min) | A (%) | B (%) |
| Gradient | 0 | 70 | 30 |
| | 2 | 70 | 30 |
| | 27 | 45 | 55 |
| | 40 | 20 | 80 |
| | 45 | 20 | 80 |
| | 46 | 70 | 30 |
| | 56 | 70 | 30 |

Ion chromatography (IC)

| | |
|---|---|
| Instrument | Metrohm 940 professional IC |
| Sample center | 889 IC |
| Detector | conductivity detector |
| Eluent (anion) | 3.2 mmol/L Na₂CO₃ + 1.0 mmol/L NaHCO₃ |
| Suppressor | 0.5% H₂SO₄ |
| solution | |
| Column | Anion A SUPP 5-150 or Cation Column C4-150 |
| Column | 30° C. |
| temperature | |
| Flow rate | 0.7 mL/min (anion) |
| Injection volume | 20 μL |

The free base concentration and acid ion concentration in the same sample were determined by IC, and the base:acid stoichiometric ratio in this sample was then calculated as follows:

$$free base : acid ion = \frac{{C}_{F}}{{M}_{F}} : \frac{{C}_{C}}{{M}_{C}}$$

wherein C_{F} is the free base concentration (mg/mL), M_{F} is the molar mass of the free base (g/mol), C_{C} is the acid ion concentration (mg/mL), and M_{C} is the molar mass of the acid ion (g/mol).

### Example 1: Amorphous Free Base (Sample No. Y11526-45-RV-FWD1509-AF-SU12)

About 300 mg of the compound of formula (I) (with a purity of 99.9%) were weighed and placed in a 40 mL glass bottle, and 10 mL of methanol/acetonitrile (v:v=1:1) were added to obtain a clear solution. The clear solution was subjected to rapid volatilization (i.e., rotary evaporation) to remove the solvent to afford about 290 mg of an off-white solid in about 97% yield.

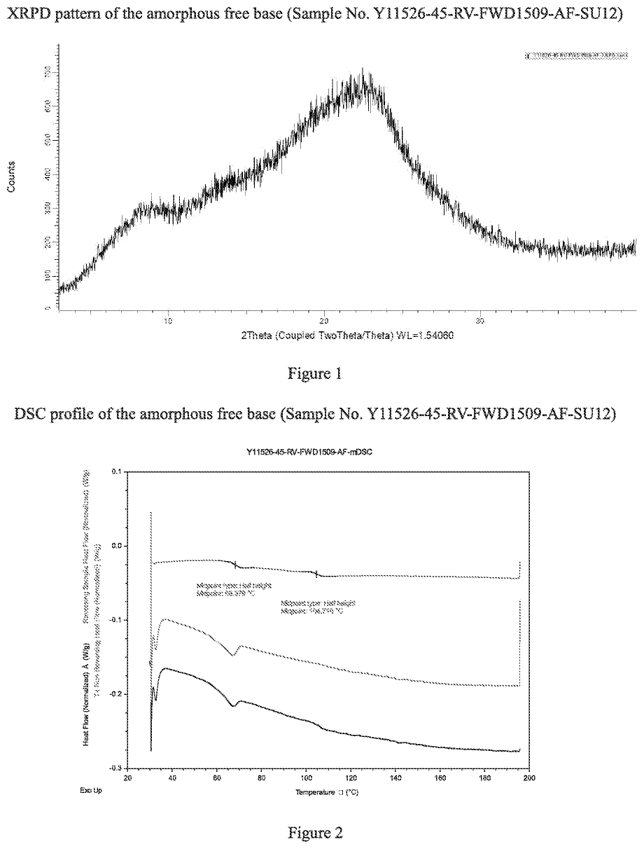

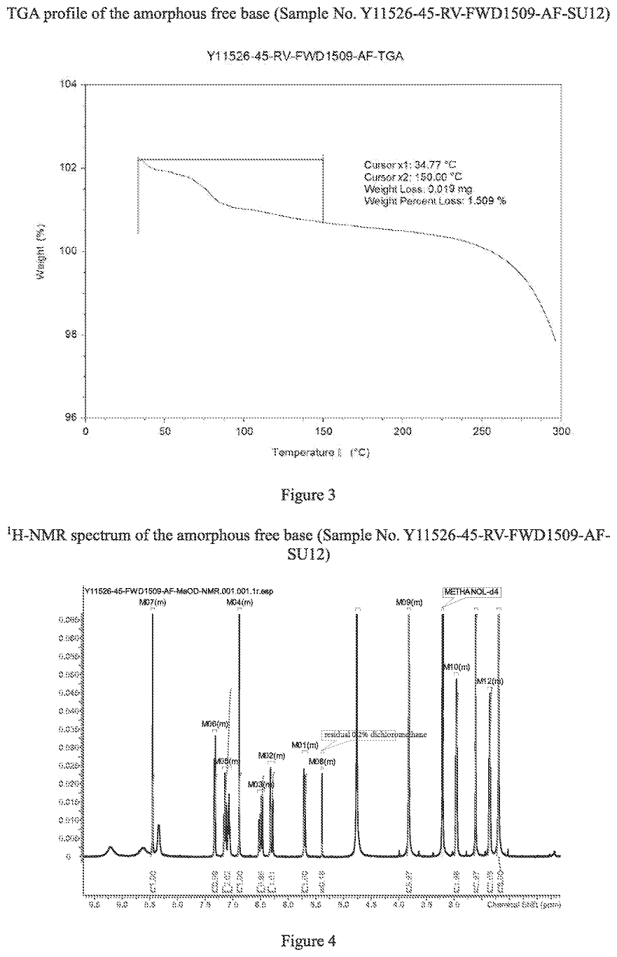

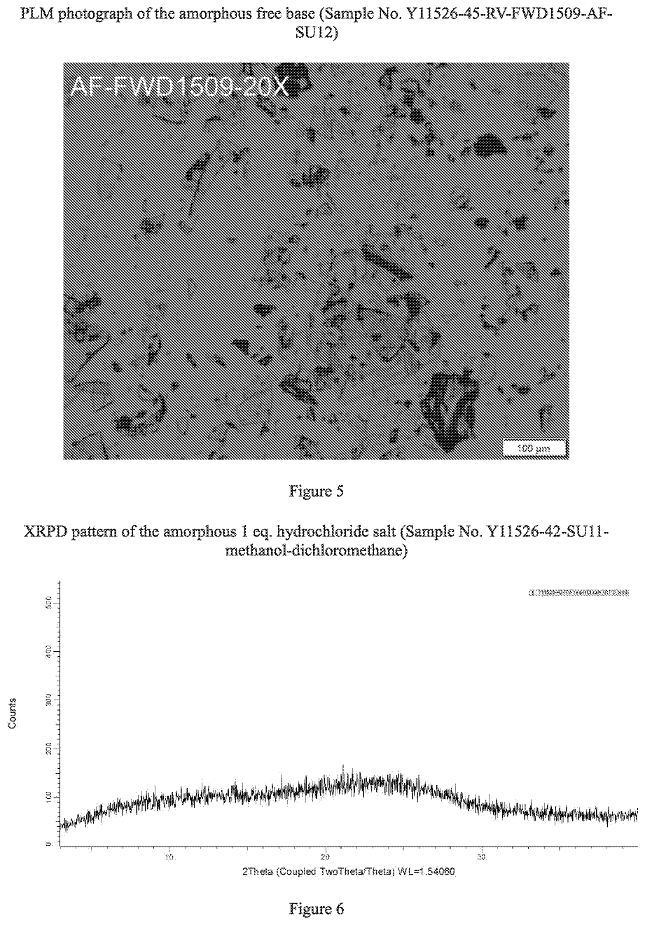

HPLC showed that the product had a purity of 99.9%. PLM showed that the product was an irregular sample (FIG. 5). XRPD showed that the product was amorphous (FIG. 1). DSC showed that the product had a glass-transition temperature of 68.4° C. and 104.7° C. (FIG. 2). TGA showed that the product had a weight loss of about 5.1% at 150° C. (FIG. 3).

The amorphous free base (Sample No. Y11526-45-RV-FWD1509-AF-SU12) was chemically and physically stable at the end of the preparation.

### Example 2: Amorphous 1 Eq. Hydrochloride Salt (Sample No. Y11526-42-SU11-Methanol-Dichloromethane)

About 300 mg of the compound of formula (I) (with a purity of 99.9%) were weighed and placed in a 40 mL glass bottle, 20 mL of methanol/dichloromethane (v:v=1:1) and 1 eq. of a diluted hydrochloric acid solution (518 μL, 44 mg/mL, in methanol/dichloromethane (v:v=1:1)) were added, and the reaction was performed at 50° C. for 2 h to obtain a clear solution. The clear solution was subjected to rapid volatilization to remove the solvent to afford about 306 mg of a pale yellow solid in about 95% yield.

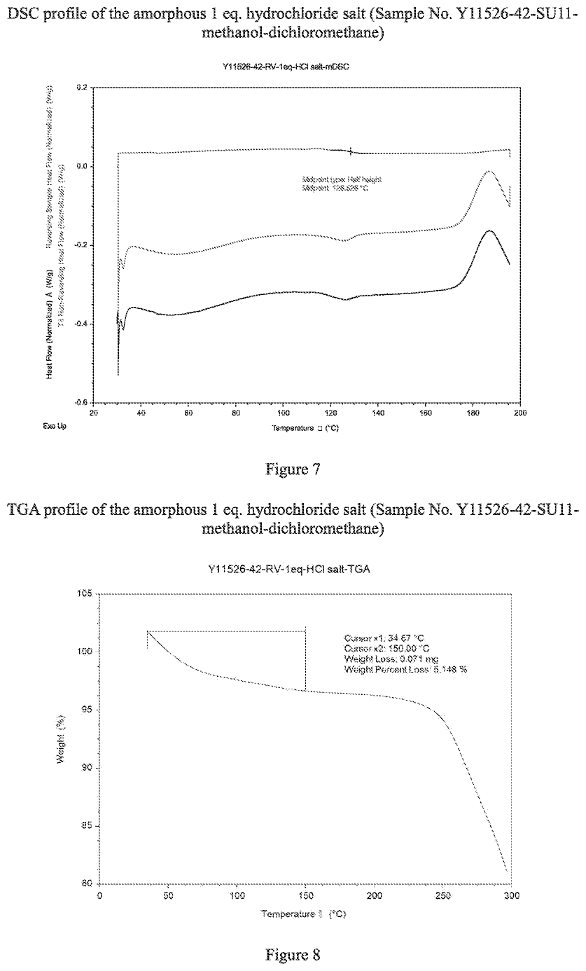

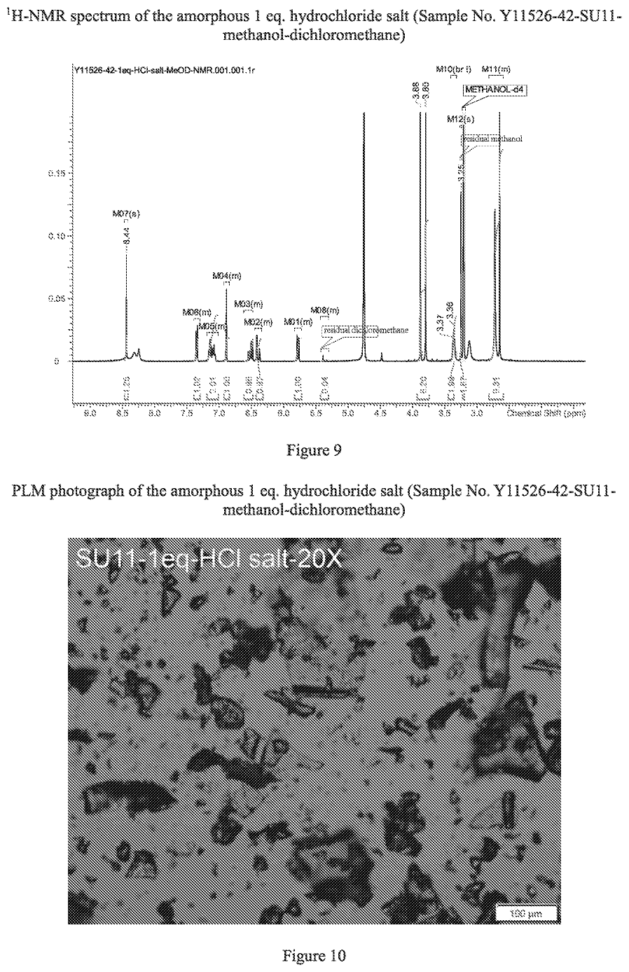

HPLC showed that the product had a purity of 99.9%. PLM showed that the product was an irregular sample (FIG. 10). XRPD showed that the product was amorphous (FIG. 6). DSC showed that the product had a glass-transition temperature of 128.5° C. (FIG. 7). TGA showed that the product had a weight loss of about 5.1% at 150° C. (FIG. 8). IC showed that the product had a free base concentration of 0.5 mg/mL and a chloride ion concentration of 33.7 mg/L, so the base:acid stoichiometric ratio in the product was about 1:1.

The amorphous 1 eq. hydrochloride salt (Sample No. Y11526-42-SU11-methanol-dichloromethane) was chemically and physically stable and reasonable in terms of the base:acid stoichiometric ratio at the end of the preparation.

### Example 3: Amorphous 2 Eq. Hydrochloride Salt (Sample No. Y11526-28-SU5-Methanol-Dichloromethane)

About 300 mg of the compound of formula (I) (with a purity of 99.9%) were weighed and placed in a 40 mL glass bottle, 5 mL of methanol/dichloromethane (v:v=1:1) and 2 eq. of a diluted hydrochloric acid solution (1010 μL, 44 mg/mL, in methanol/dichloromethane (v:v=1:1)) were added, and the reaction was performed at 50° C. for 2 h, then cooled down to 25° C. and continued at this temperature for about 2 h to obtain a clear solution. The resulting clear solution was subjected to rapid volatilization to remove the solvent to afford about 310 mg of a yellow solid in about 90% yield.

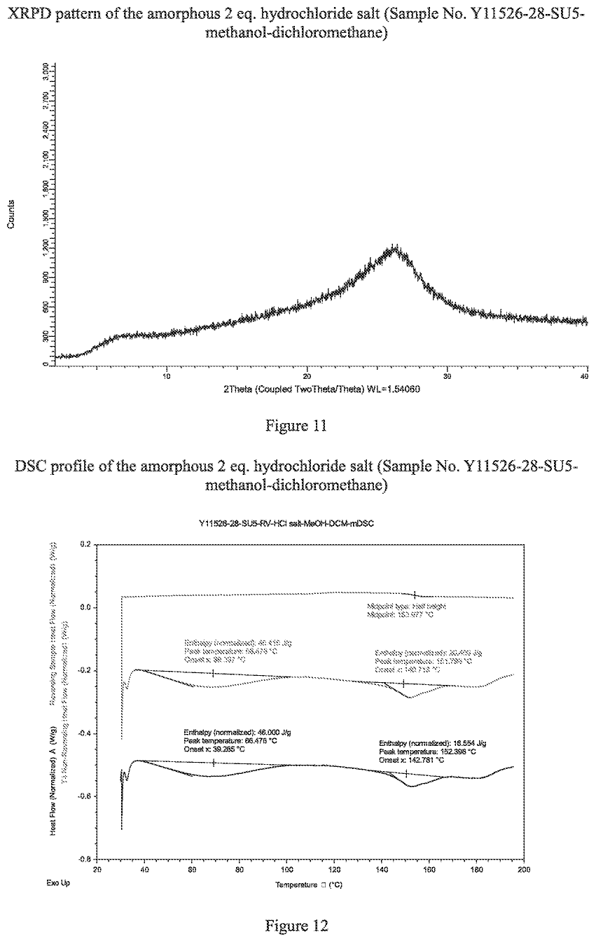

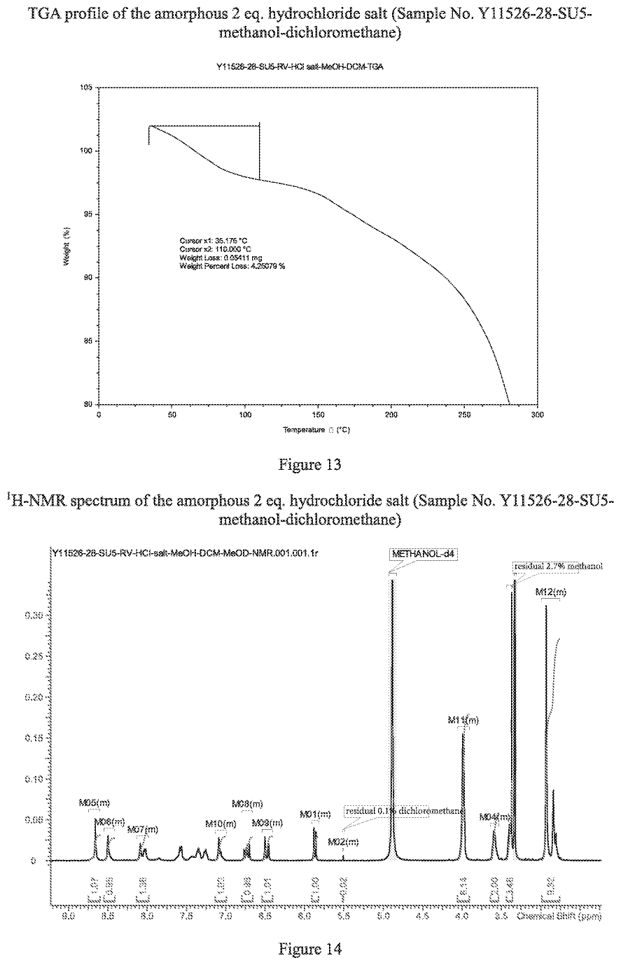

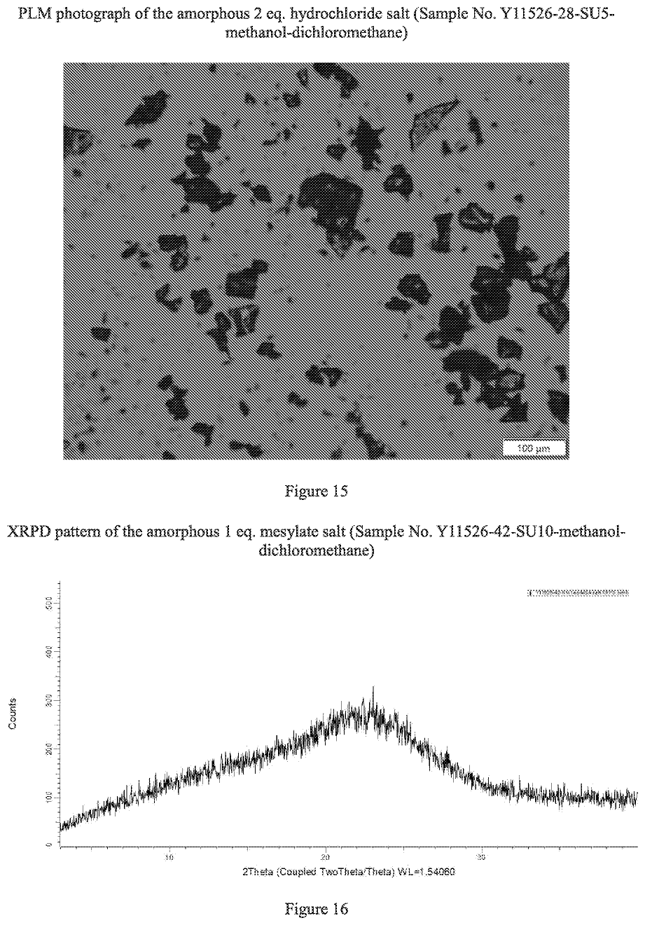

HPLC showed that the product had a purity of 99.9%. PLM showed that the product was an irregular sample (FIG. 15). XRPD showed that the product was amorphous (FIG. 11). DSC showed that the product had a glass-transition temperature of 154.0° C. (FIG. 12). TGA showed that the product had a weight loss of about 4.3% at 110° C. (FIG. 13). IC showed that the product had a free base concentration of 0.5 mg/mL and a chloride ion concentration of 77.4 mg/L, so the base:acid stoichiometric ratio in the product was about 1:2. The amorphous 2 eq. hydrochloride salt (Sample No. Y11526-28-SU5-methanol-dichloromethane) was chemically and physically stable and reasonable in terms of the base:acid stoichiometric ratio at the end of the preparation.

### Example 4: Amorphous 1 Eq. Mesylate Salt (Sample No. Y11526-42-SU10-Methanol-Dichloromethane)

The weighed compound of formula (I) was added to acetone (11.70 times by weight with respect to that of the compound of formula (I) weighed) under stirring, and the temperature was increased to 45-55° C. After dissolving, the reaction mixture was filtered while hot, the filtrate was heated to 45-55° C., water (1 time by weight with respect to that of the compound of formula (I) weighed) was added, stirring was continued at 45-55° C., methanesulfonic acid (0.188 times by weight with respect to that of the compound of formula (I) weighed) was added dropwise within 30 min, and the mixture was stirred at 45-55° C. for 60±10 min. The reaction mixture was cooled down to 20-30° C. within 1.0-2.0 h, and crystallization was performed at 10-30° C. under stirring for 1.0-2.0 h. The reaction mixture was filtered, and the filter cake was rinsed twice with acetone (2*0.78 times by weight with respect to that of the compound of formula (I) weighed). The filter cake was dried to constant weight under the conditions of 40±5° C. and ≤−0.07 MPa to obtain a high-crystallinity material.

About 300 mg of the high-crystallinity material obtained were weighed, and about 50 mL of methanol/dichloromethane (v:v=1:1) were added to obtain a clear solution. The clear solution was subjected to rapid volatilization to remove the solvent to afford about 268 mg of a pale yellow solid in about 72% yield.

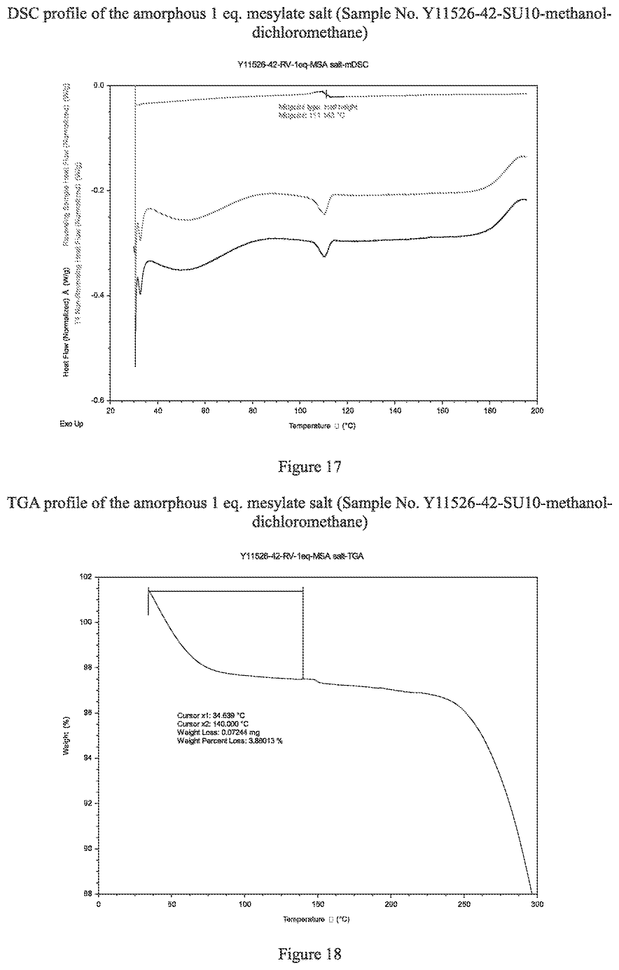

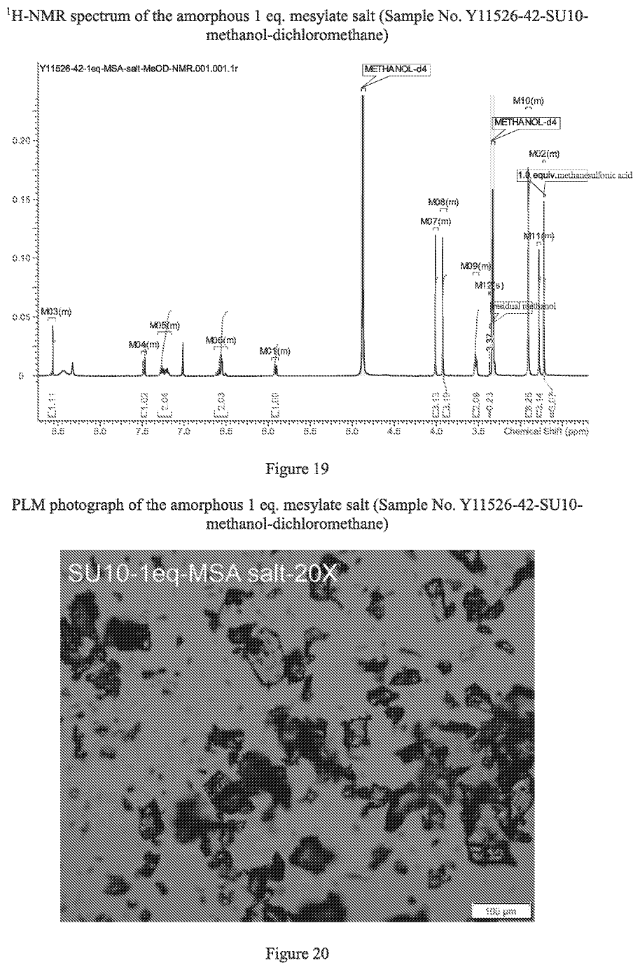

HPLC showed that the product had a purity of 99.9%. PLM showed that the product was an irregular sample (FIG. 20). XRPD showed that the product was amorphous (FIG. 16). DSC showed that the product had a glass-transition temperature of 111.1° C. (FIG. 17). TGA showed that the product had a weight loss of about 3.9% at 140° C. (FIG. 18). ¹H-NMR showed a base:acid stoichiometric ratio of about 1:1 in the product (FIG. 19).

The amorphous 1 eq. mesylate salt (Sample No. Y11526-42-SU10-methanol-dichloromethane) was chemically and physically stable and reasonable in terms of the base:acid stoichiometric ratio at the end of the preparation.

### Example 5: Amorphous 2 Eq. Mesylate Salt (Sample No. Y11526-28-SU4-Methanol-Dichloromethane)

About 300 mg of the compound of formula (I) (with a purity of 99.9%) were weighed and placed in a 40 mL glass bottle, 2 mL of methanol and 2 eq. of a diluted methanesulfonic acid solution (798 μL, 148 mg/mL, in methanol) were added, and the clear solution became a yellow opaque system. 2 mL of dichloromethane were added, the solution became clear, and 1 mL of methanol/dichloromethane (v:v=1:1) was continued to be added. The reaction was performed at 50° C. for 2 h, then cooled down to 25° C. and continued at this temperature for about 3 h to obtain a clear solution. The resulting clear solution was subjected to rapid volatilization to remove the solvent to afford about 380 mg of a yellow solid in about 85% yield.

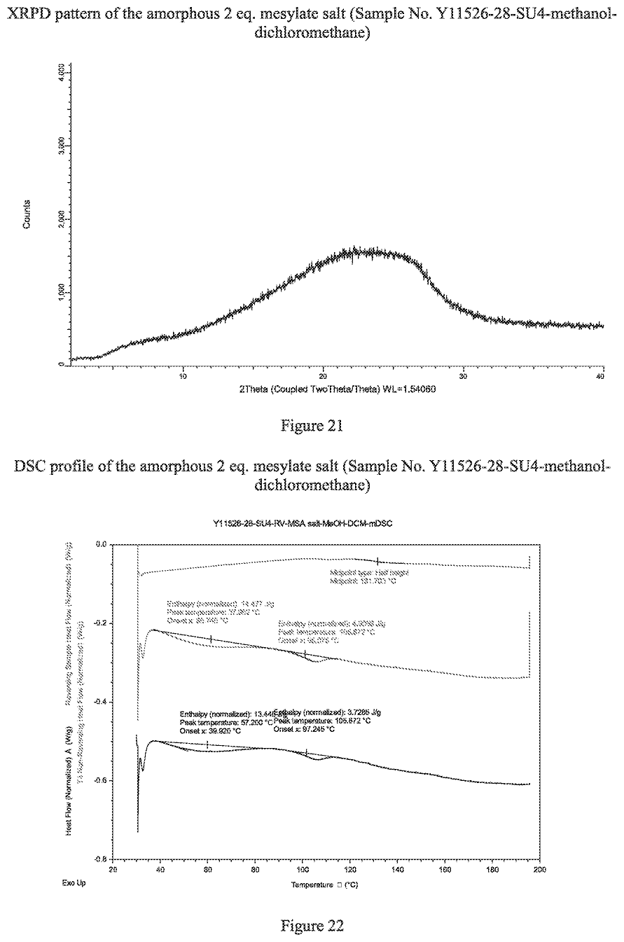

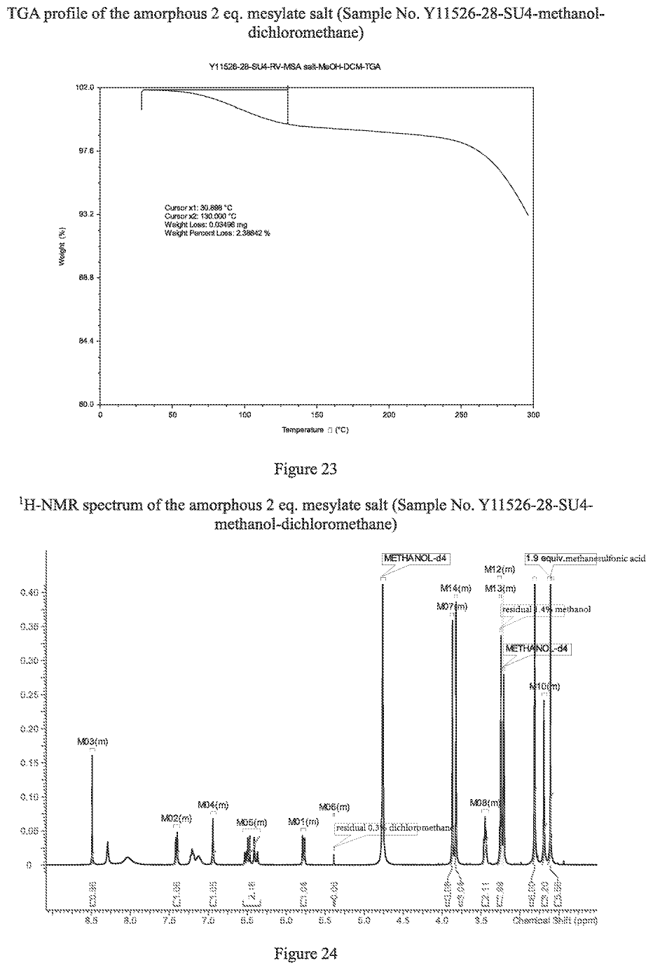

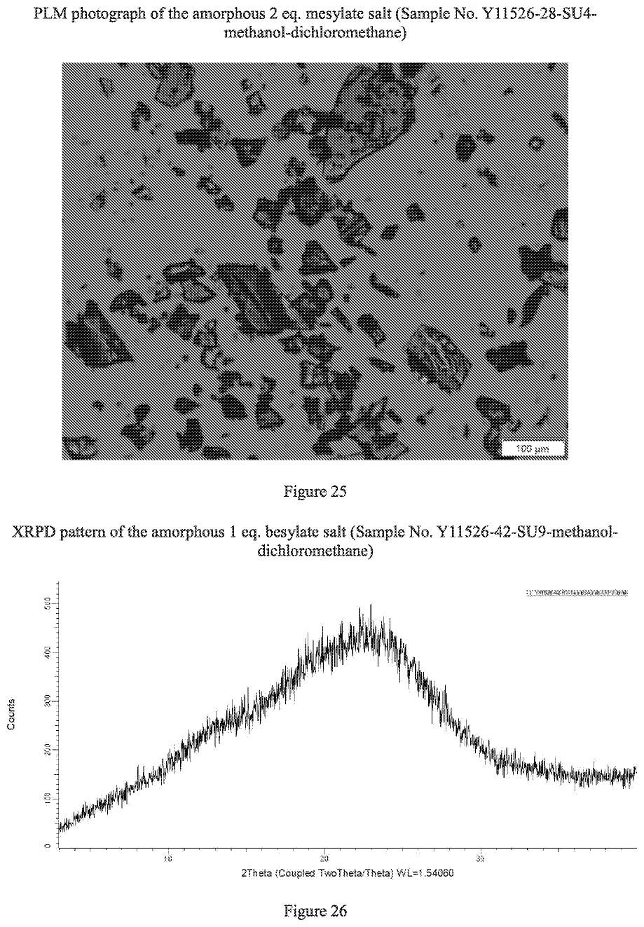

HPLC showed that the product had a purity of 99.9%. PLM showed that the product was an irregular sample (FIG. 25). XRPD showed that the product was amorphous (FIG. 21). DSC showed that the product had a glass-transition temperature of 131.7° C. (FIG. 22). TGA showed that the product had a weight loss of about 2.4% at 130° C. (FIG. 23). ¹H-NMR showed a base:acid stoichiometric ratio of about 1:2 in the product (FIG. 24).

The amorphous 2 eq. mesylate salt (Sample No. Y11526-28-SU4-methanol-dichloromethane) was chemically and physically stable and reasonable in terms of the base:acid stoichiometric ratio at the end of the preparation.

### Example 6: Amorphous 1 Eq. Besylate Salt (Sample No. Y11526-42-SU9-Methanol-Dichloromethane)

About 300 mg of the compound of formula (I) (with a purity of 99.9%) were weighed and placed in a 250 mL round-bottom flask together with 1 eq. of benzenesulfonic acid, 15 mL of water were added, and the reaction was performed at 50° C. for 2 h to obtain a clear solution followed by solid precipitation. The suspension was pre-frozen in a dry ice/ethanol mixture for 2 h, and then water was removed by freeze drying to afford a low-crystallinity sample. The freeze-dried sample was dissolved in 5 mL of methanol/dichloromethane (v:v=1:1) to obtain a clear solution. The clear solution was subjected to rapid volatilization to remove the solvent to afford about 320 mg of a pale yellow solid in about 83% yield.

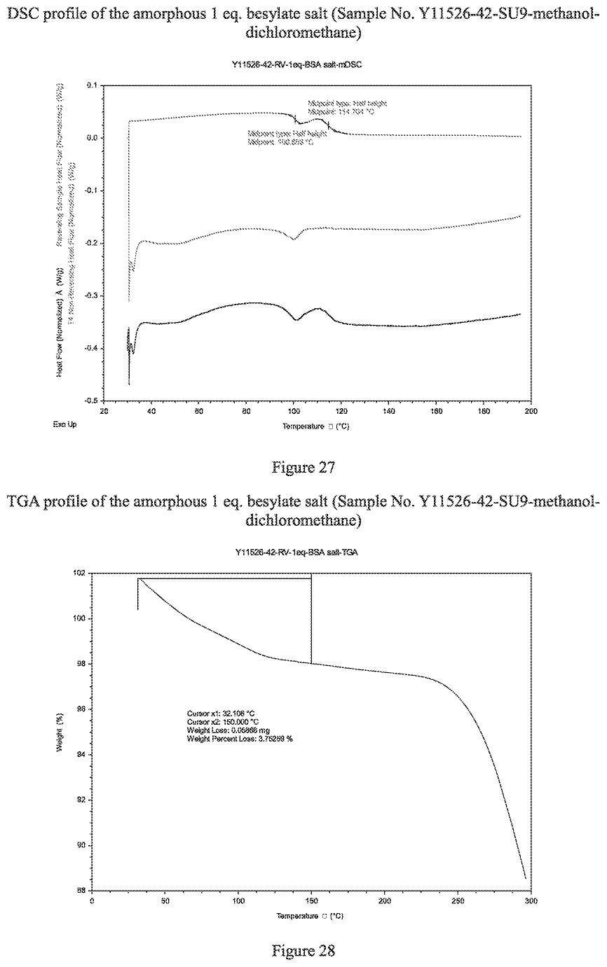

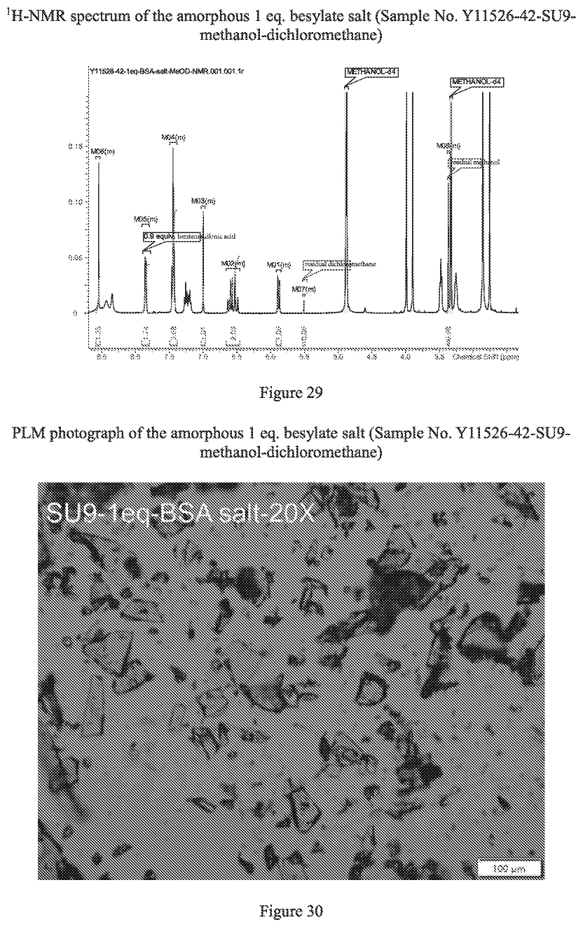

HPLC showed that the product had a purity of 99.7%. PLM showed that the product was an irregular sample (FIG. 30). XRPD showed that the product was amorphous (FIG. 26). DSC showed that the product had a glass-transition temperature of 100.7° C. and 114.7° C. (FIG. 27). TGA showed that the product had a weight loss of about 3.8% at 150° C. (FIG. 28). ¹H-NMR showed a base:acid stoichiometric ratio of about 1:1 in the product (FIG. 29).

The amorphous 1 eq. besylate salt (Sample No. Y11526-42-SU9-methanol-dichloromethane) was chemically and physically stable and reasonable in terms of the base:acid stoichiometric ratio at the end of the preparation.

### Example 7: Amorphous 2 Eq. Besylate Salt (Sample No. Y11526-30-SU1-Water)

About 300 mg of the compound of formula (I) (with a purity of 99.9%) were weighed and placed in a 250 mL round-bottom flask together with 2 eq. of benzenesulfonic acid, 15 mL of water were added, and the reaction was performed at 50° C. for 2 h to obtain a clear solution. The resulting clear solution was pre-frozen in a dry ice/ethanol mixture for 2 h, and then water was removed by freeze drying to afford about 380 mg of a yellow solid in about 81% yield.

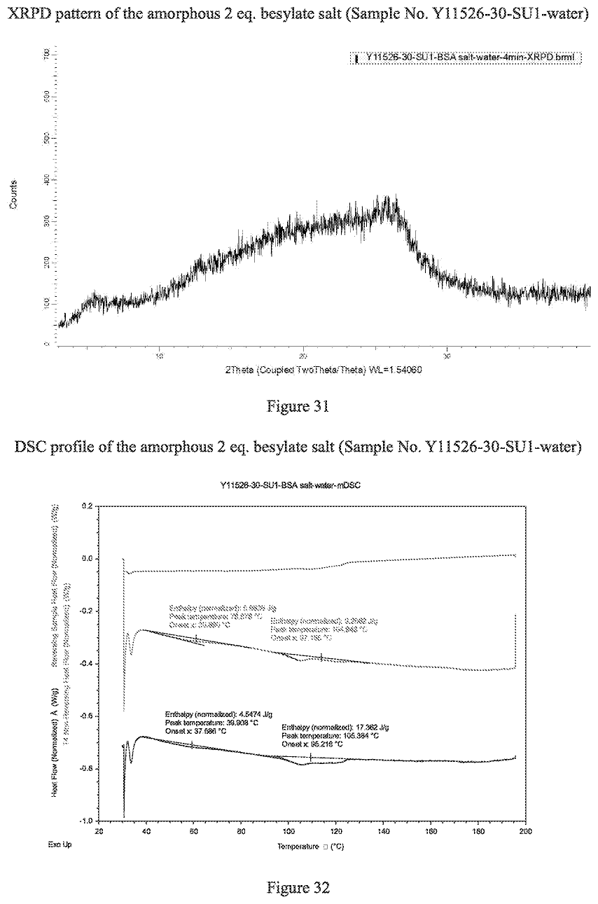

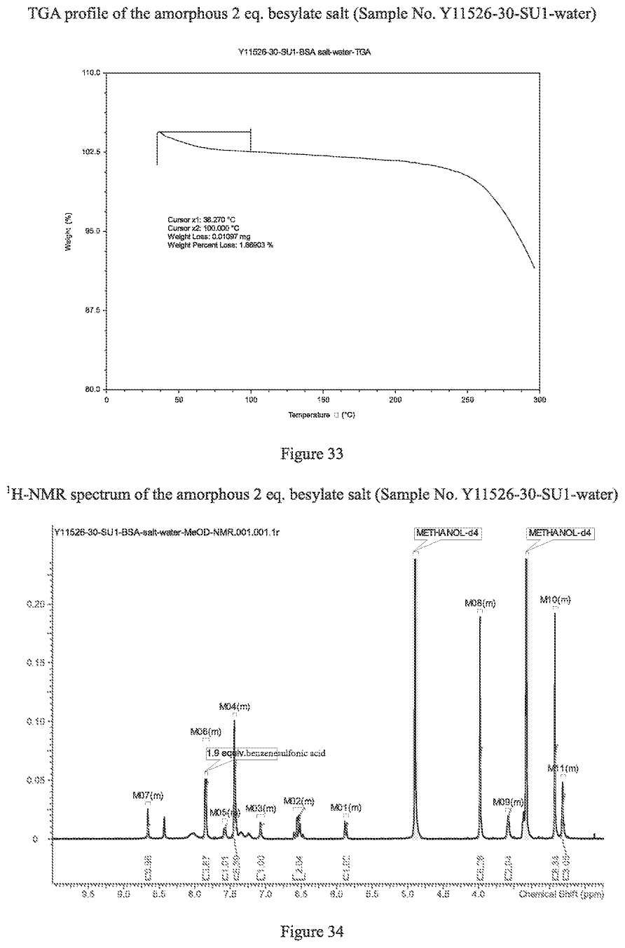

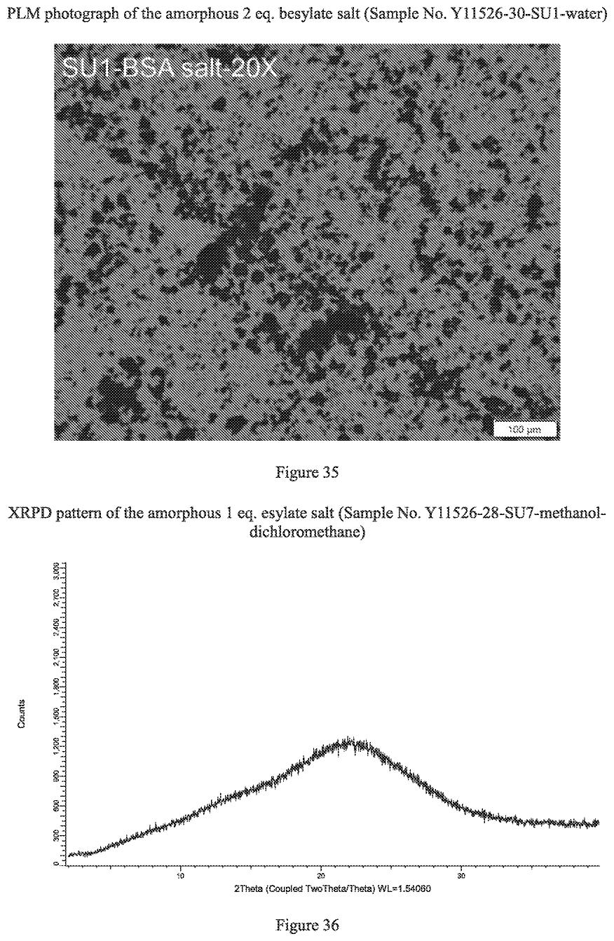

HPLC showed that the product had a purity of 99.7%. PLM showed that the product was an irregular sample (FIG. 35). XRPD showed that the product was amorphous (FIG. 31). DSC showed that the product had no glass-transition temperature (FIG. 32). TGA showed that the product had a weight loss of about 1.9% at 100° C. (FIG. 33). ¹H-NMR showed a base:acid stoichiometric ratio of about 1:2 in the product (FIG. 34).

The amorphous 2 eq. besylate salt (Sample No. Y11526-30-SU1-water) was chemically and physically stable and reasonable in terms of the base:acid stoichiometric ratio at the end of the preparation.

### Example 8: Amorphous 1 Eq. Esylate Salt (Sample No. Y11526-28-SU7-Methanol-Dichloromethane)

About 300 mg of the compound of formula (I) (with a purity of 99.9%) were weighed and placed in a 40 mL glass bottle, 5 mL of methanol/dichloromethane (v:v=1:1) and 1 eq. of a diluted ethanesulfonic acid solution (541 μL, 128 mg/mL, in methanol/dichloromethane (v:v=1:1)) were added, and the reaction was performed at 50° C. for 2 h, then cooled down to 25° C. and continued at this temperature for about 2 h to obtain a clear solution. The resulting clear solution was subjected to rapid volatilization to remove the solvent to afford about 340 mg of a yellow solid in about 90% yield.

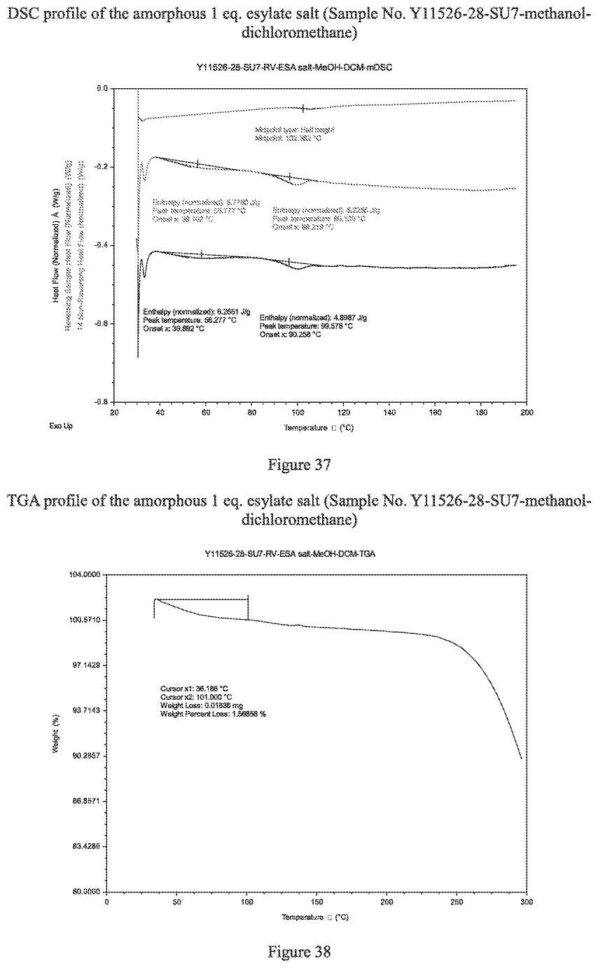

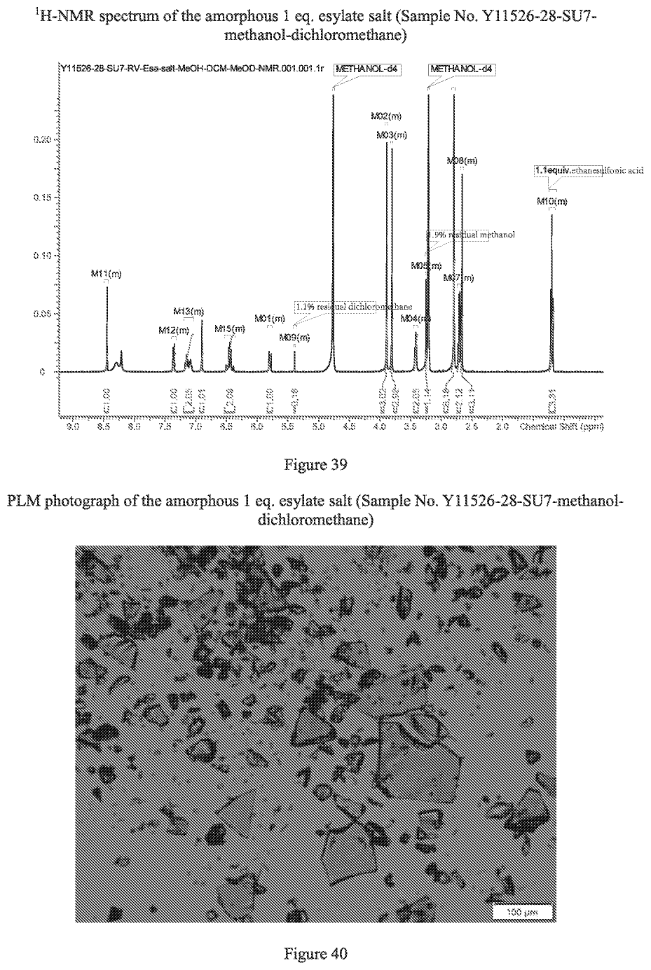

HPLC showed that the product had a purity of 99.9%. PLM showed that the product was an irregular sample (FIG. 40). XRPD showed that the product was amorphous (FIG. 36). DSC showed that the product had a glass-transition temperature of 102.4° C. (FIG. 37). TGA showed that the product had a weight loss of about 1.6% at 101° C. (FIG. 38). ¹H-NMR showed a base:acid stoichiometric ratio of about 1:1 in the product (FIG. 39).

The amorphous 1 eq. esylate salt (Sample No. Y11526-28-SU7-methanol-dichloromethane) was chemically and physically stable and reasonable in terms of the base:acid stoichiometric ratio at the end of the preparation.

### Example 9: Amorphous 1 Eq. Maleate Salt (Sample No. Y11526-30-SU2-Water)

About 300 mg of the compound of formula (I) (with a purity of 99.9%) were weighed and placed in a 250 mL round-bottom flask together with 1 eq. of maleic acid, 20 mL of water were added, and the reaction was performed at 50° C. for 2 h to obtain a clear solution. The resulting clear solution was pre-frozen in a dry ice/ethanol mixture for 2 h, and then water was removed by freeze drying to afford about 330 mg of a pale yellow solid in about 89% yield.

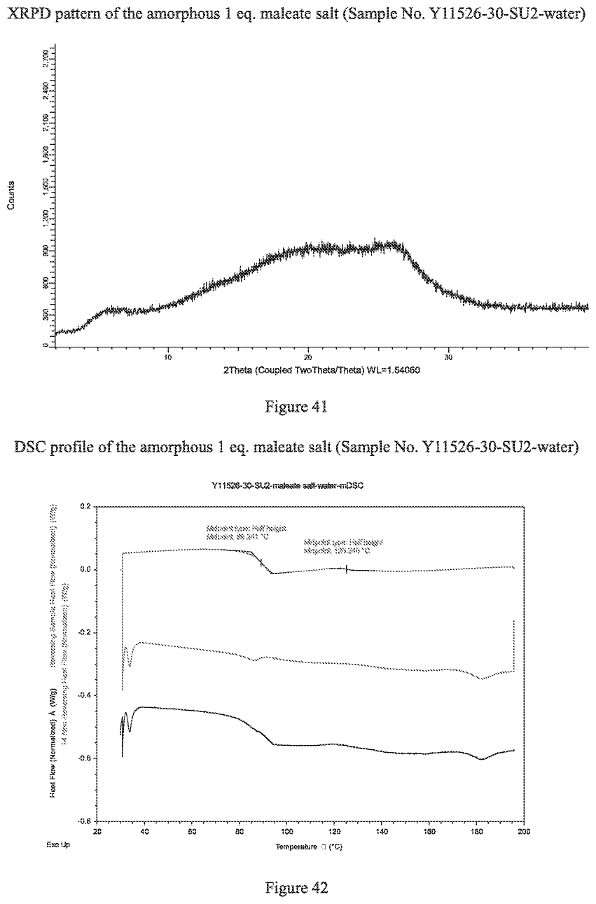

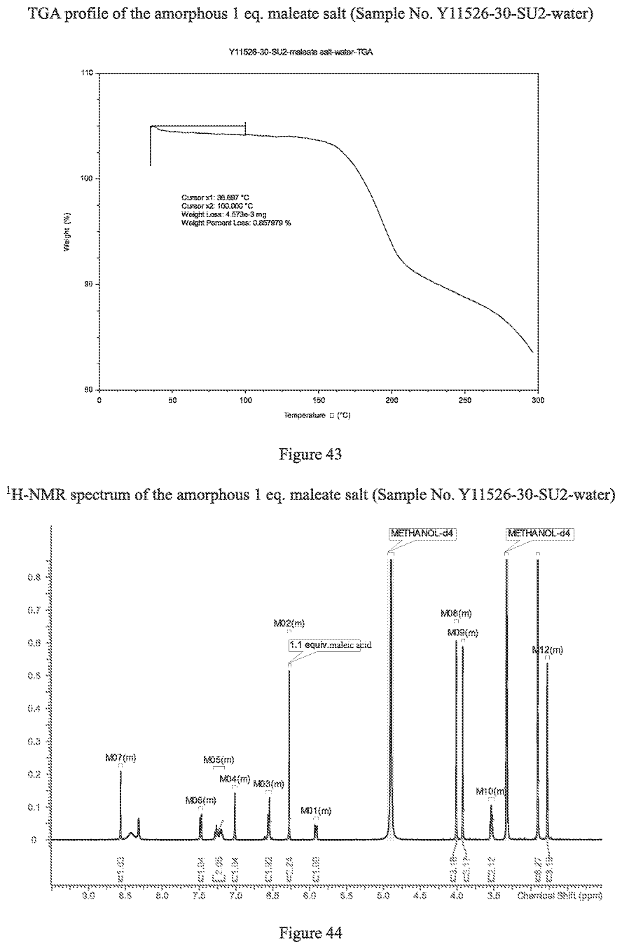

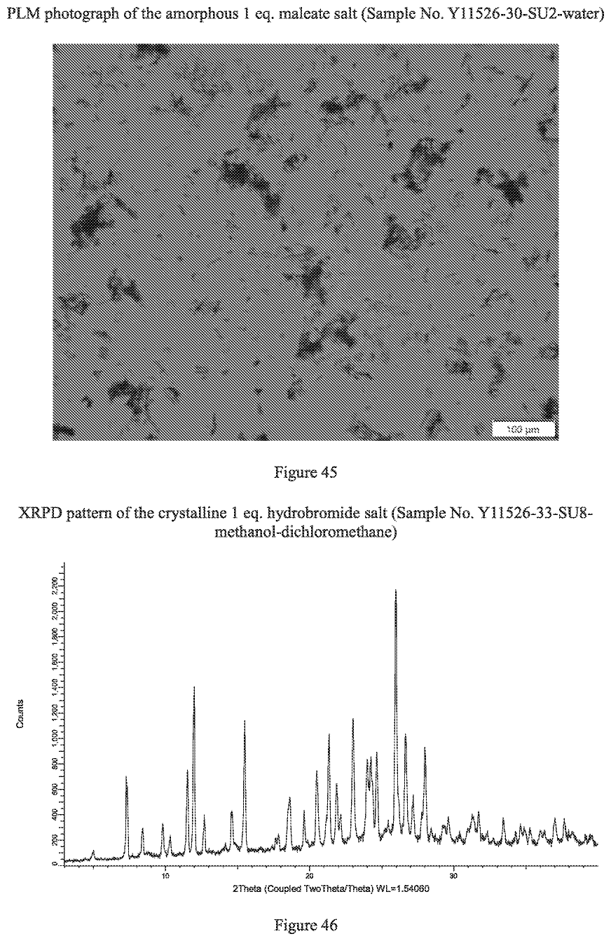

HPLC showed that the product had a purity of 99.9%. PLM showed that the product was an irregular sample (FIG. 45). XRPD showed that the product was amorphous (FIG. 41). DSC showed that the product had a glass-transition temperature of 89.2° C. and 125.2° C. (FIG. 42). TGA showed that the product had a weight loss of about 0.9% at 100° C. (FIG. 43). ¹H-NMR showed a base:acid stoichiometric ratio of about 1:1 in the product (FIG. 44).

The amorphous 1 eq. maleate salt (Sample No. Y11526-30-SU2-water) was chemically and physically stable and reasonable in terms of the base:acid stoichiometric ratio at the end of the preparation.

### Example 10: The Crystalline 1 Eq. Hydrobromide Salt (Sample No. Y11526-33-SU8-Methanol-Dichloromethane)

About 300 mg of the compound of formula (I) (with a purity of 99.9%) were weighed and placed in a 40 mL glass bottle, 20 mL of methanol/dichloromethane (v:v=1:1) and 1 eq. of a diluted hydrobromic acid solution (728 μL, 70 mg/mL, in methanol/dichloromethane (v:v=1:1)) were added, and the reaction was performed at 50° C. for 2 h to obtain a nearly clear solution (trace insoluble impurities). The impurities were filtered through a 0.45 μm filter membrane to obtain a clear solution, and the resulting clear solution was subjected to rapid volatilization to remove the solvent to afford about 310 mg of a pale yellow solid in about 90% yield.

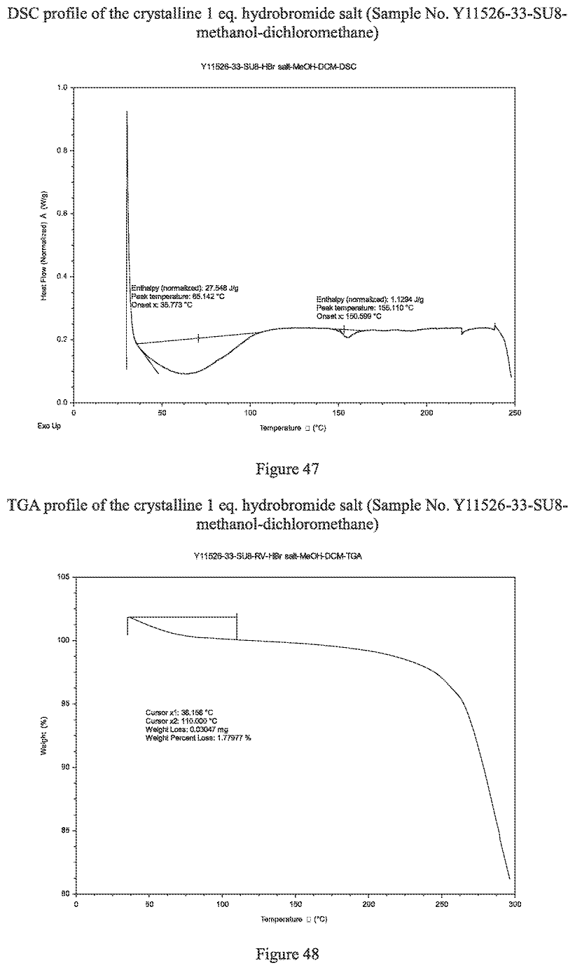

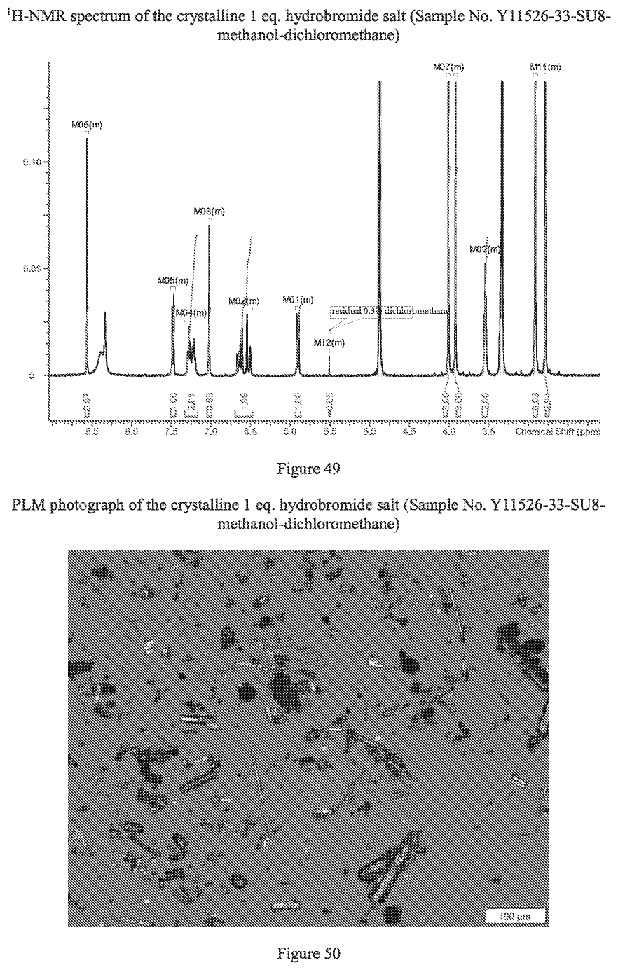

HPLC showed that the product had a purity of 99.9%. PLM showed that the product as rod-like and block-like samples (FIG. 50). XRPD showed that the product had a high crystallinity (FIG. 46). DSC showed that the product started to dehydrate from about 30° C. (FIG. 47). TGA showed that the product had a weight loss of about 1.8% at 110° C. (FIG. 48). KF showed that the product contained 1.9% of water. IC showed that the product had a free base concentration of 0.5 mg/mL and a bromide ion concentration of 68.1 mg/L, so the base:acid stoichiometric ratio in the product was about 1:1.

The crystalline 1 eq. hydrobromide salt (Sample No. Y11526-33-SU8-methanol-dichloromethane) was chemically and physically stable and reasonable in terms of the base:acid stoichiometric ratio at the end of the preparation.

### Example 11: Amorphous Nitrate Salt (Sample No. Y1526-18-RV7-Methanol-Dichloromethane)

About 100 mg of the compound of formula (I) (with a purity of 99.9%) were weighed and placed in a 40 mL glass bottle, 10 mL of 1,4-dioxane were added, 2 eq. of a diluted nitric acid solution (144 μL, 180 mg/mL, in 1,4-dioxane) were added, and the reaction was performed at 50° C. for 2 h. The resulting clear solution was pre-frozen in a dry ice/ethanol mixture for 2 h, and then 1,4-dioxane was removed by freeze drying to afford a nearly amorphous material.

About 80 mg of the nearly amorphous material were weighed and placed in a 40 mL glass bottle, and 10 mL of methanol/dichloromethane (v:v=1:1) were added to obtain a clear solution. The clear solution was subjected to rapid volatilization to remove the solvent to afford the amorphous nitrate salt.

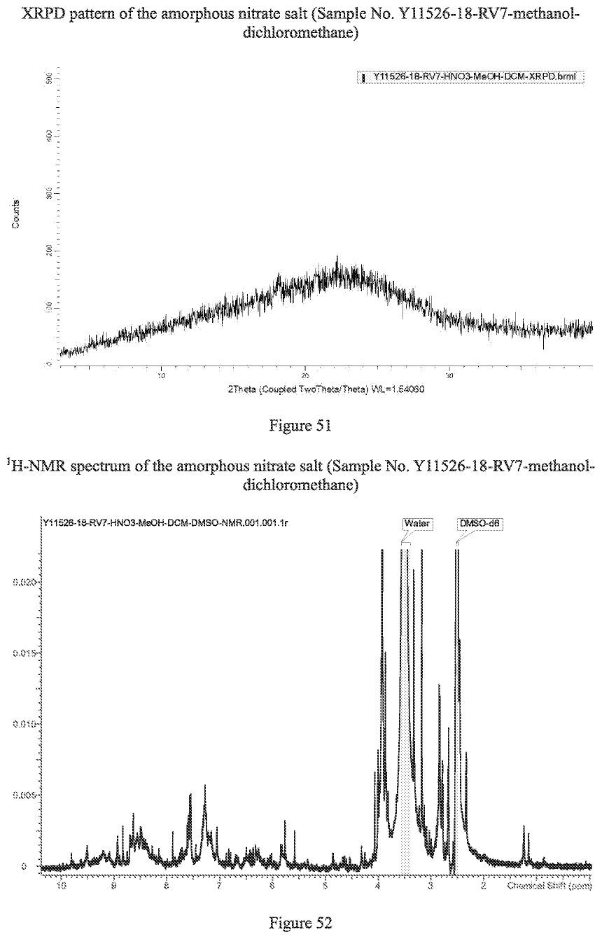

XRPD showed that the product was amorphous (FIG. 51). ¹H-NMR showed that the product was degraded (FIG. 52).

The amorphous nitrate salt (Sample No. Y11526-18-RV7-methanol-dichloromethane) was at least chemically unstable at the end of the preparation.

### Example 12: Amorphous Sulfate Salt (Sample No. Y11526-15-RV3-Methanol-Acetonitrile)

About 100 mg of the compound of formula (I) (with a purity of 99.9%) were weighed and placed in a 40 mL glass bottle, 10 mL of 1,4-dioxane were added, 2 eq. of a diluted sulfuric acid solution (228 μL, 180.3 mg/mL, in 1,4-dioxane) were added, and the reaction was performed at 50° C. for 2 h. The resulting clear solution was pre-frozen in a dry ice/ethanol mixture for 2 h, and then 1,4-dioxane was removed by freeze drying to afford a nearly amorphous material.

About 80 mg of the nearly amorphous material obtained were weighed and placed in a 40 mL glass bottle, and about 10 mL of methanol/acetonitrile (v:v=1:1) were added to obtain a clear solution. The clear solution was subjected to rapid volatilization to remove the solvent to afford the amorphous sulfate salt.

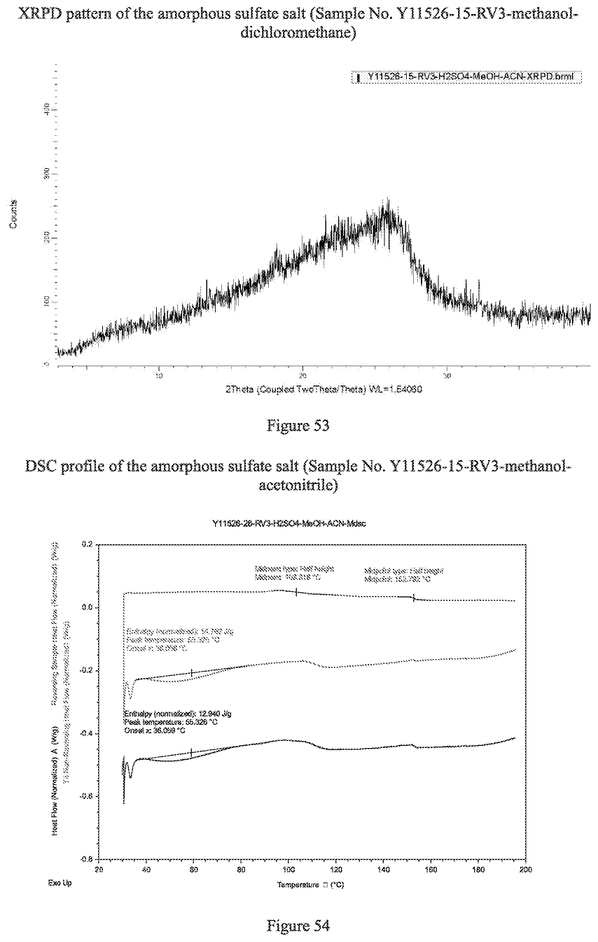

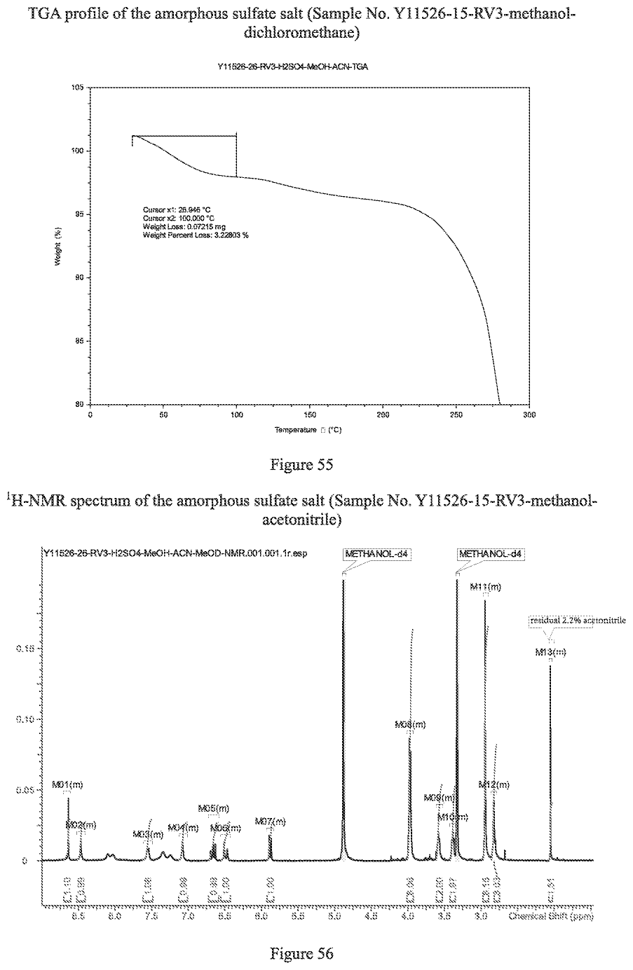

HPLC showed that the product had a purity of 99.7%. PLM showed that the product was an irregular sample (FIG. 57). XRPD showed that the product was amorphous (FIG. 53). DSC showed that the product had a glass-transition temperature of 103.3° C. and 152.8° C. (FIG. 54). TGA showed that the product had a weight loss of about 3.2% at 100° C. (FIG. 55). IC showed that the product had a free base concentration of 0.5 mg/mL and a sulfate concentration of 162.5 mg/L, so the base:acid stoichiometric ratio in the product was about 1:1.7, which was unreasonable.

The amorphous sulfate salt (Sample No. Y11526-15-RV3-methanol-acetonitrile) was chemically stable at the end of the preparation, but was physically unstable due to a certain hygroscopicity and unreasonable in terms of the alkali:acid stoichiometric ratio.

### Example 13: Amorphous 2 Eq. p-Tosylate Salt (Sample No. Y11526-23-FD11-Water)

About 100 mg of the compound of formula (I) (with a purity of 99.9%) were weighed and placed in a 40 mL glass bottle together with 2 eq. of p-toluenesulfonic acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford a nearly amorphous material. The resulting nearly amorphous material was fully dissolved in 10 mL of water, and the resulting clear solution was pre-frozen in a dry ice/ethanol mixture for 2 h and then water was removed by freeze drying to afford the amorphous 2 eq. p-tosylate salt.

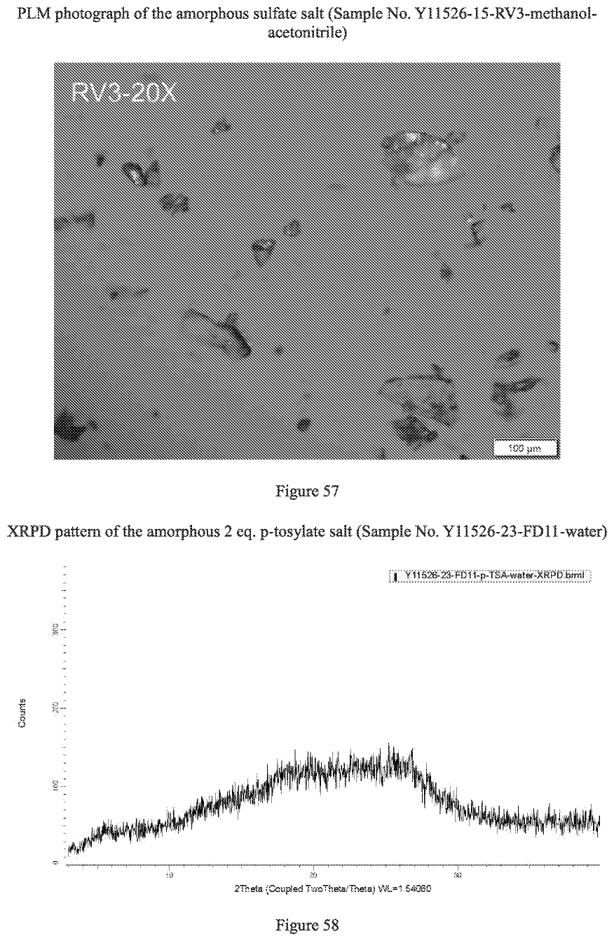

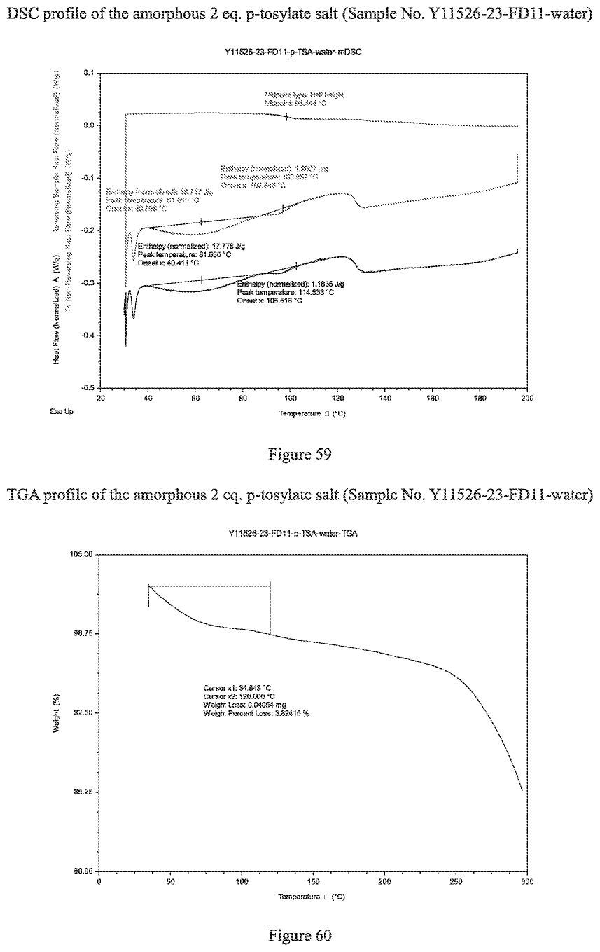

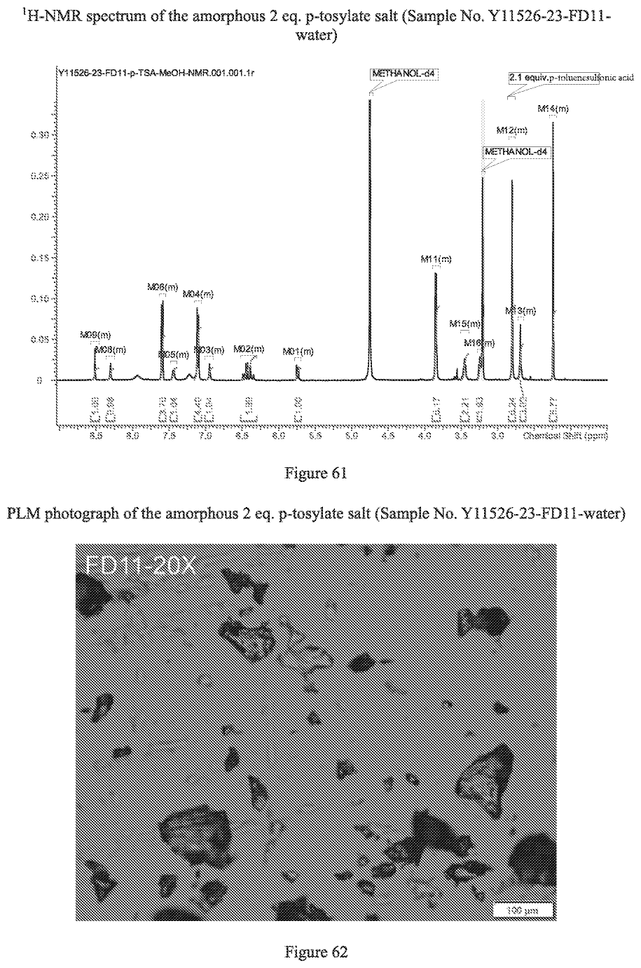

HPLC showed that the product had a purity of 99.6%. PLM showed that the product as a block-like sample (FIG. 62). XRPD showed that the product was amorphous (FIG. 58). DSC showed that the product had a glass-transition temperature of 98.4° C. (FIG. 59). TGA showed that the product had a weight loss of about 3.8% at 140° C. (FIG. 60). ¹H-NMR showed a base:acid stoichiometric ratio of about 1:2 in the product (FIG. 61).

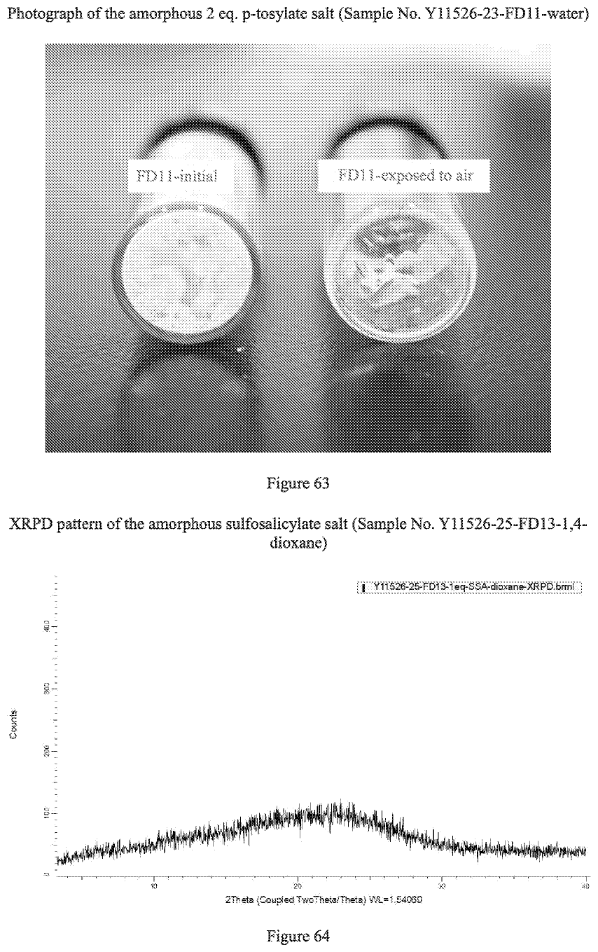

The amorphous 2 eq. p-tosylate salt (Sample No. Y11526-23-FD11-water) was chemically stable and reasonable in terms of the base:acid stoichiometric ratio at the end of the preparation, but was physically unstable due to caking and fusion to a glassy state upon short exposure to environmental conditions (FIG. 63).

### Example 14: Amorphous Sulfosalicylate Salt (Sample No. Y11526-25-FD13-1,4-Dioxane)

About 100 mg of the compound of formula (I) (with a purity of 99.9%) were weighed and placed in a 40 mL glass bottle together with 1 eq. of sulfosalicylic acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford the amorphous sulfosalicylate salt.

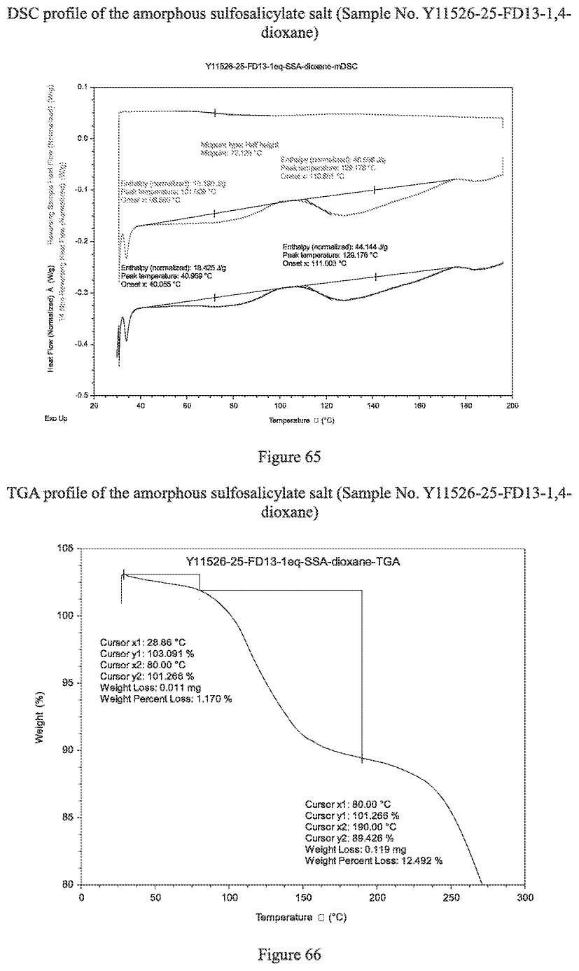

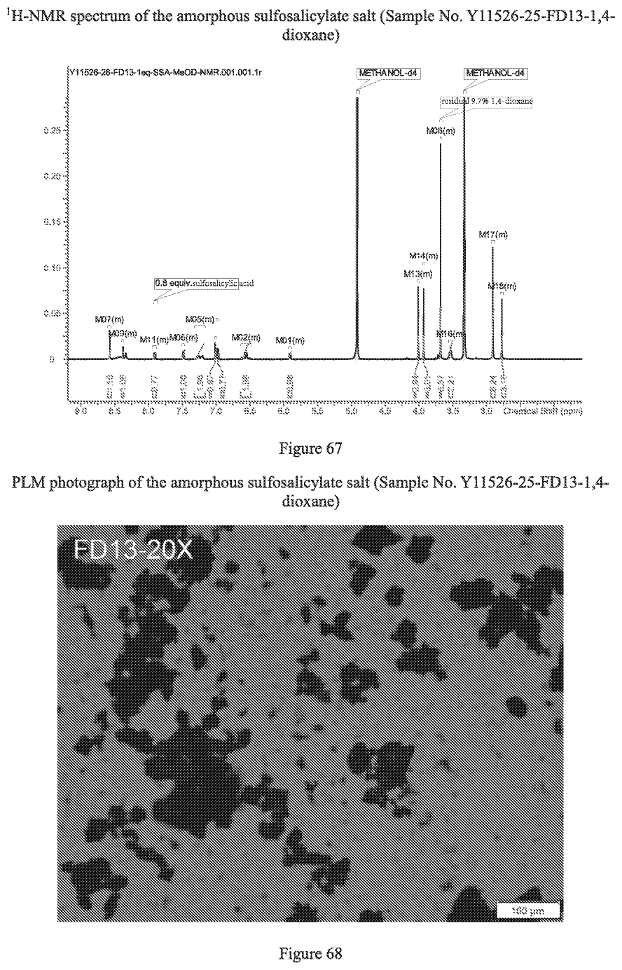

HPLC showed that the product had a purity of 99.7%. PLM showed that the product was an irregular sample (FIG. 68). XRPD showed that the product was amorphous (FIG. 64). DSC showed that the product had a glass-transition temperature of 72.1° C. (FIG. 65). TGA showed that the product had a weight loss of about 1.2% at 80° C. (FIG. 66). ¹H-NMR showed a base:acid stoichiometric ratio of about 1:0.8 in the product (FIG. 67), which was unreasonable.

The amorphous sulfosalicylate salt (Sample No. Y11526-25-FD13-1,4-dioxane) was chemically and physically stable, but unreasonable in terms of the base:acid stoichiometric ratio at the end of the preparation.

### Example 15: Amorphous Sulfosalicylate Salt (Sample No. Y11526-25-FD12-1,4-Dioxane)

About 100 mg of the compound of formula (I) (with a purity of 99.9%) were weighed and placed in a 40 mL glass bottle together with 2 eq. of sulfosalicylic acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford the amorphous sulfosalicylate salt.

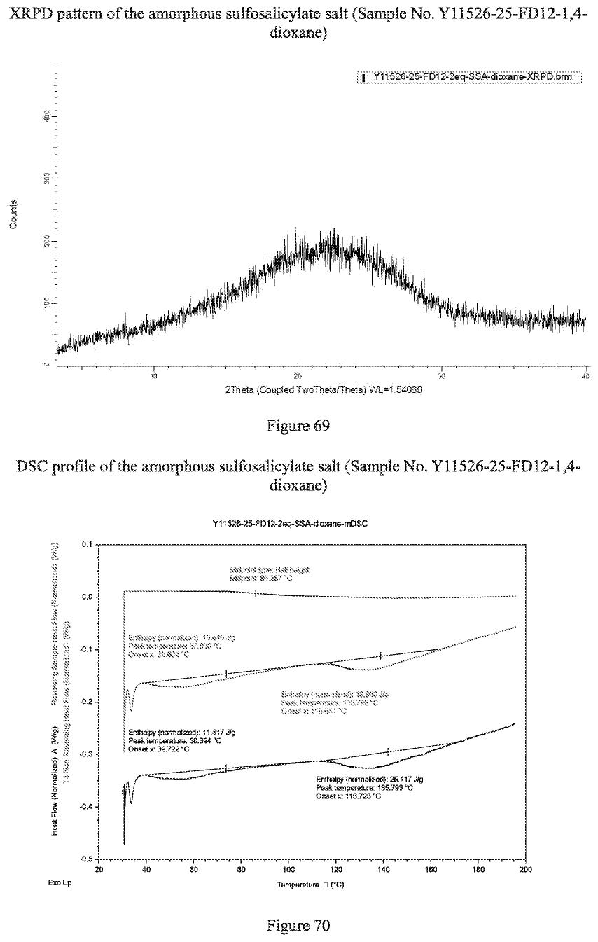

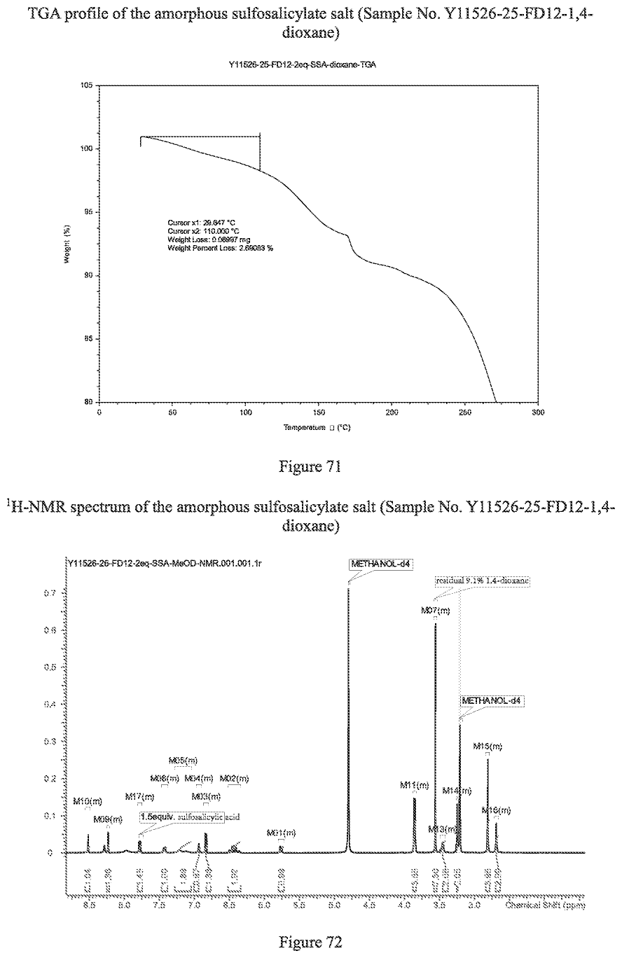

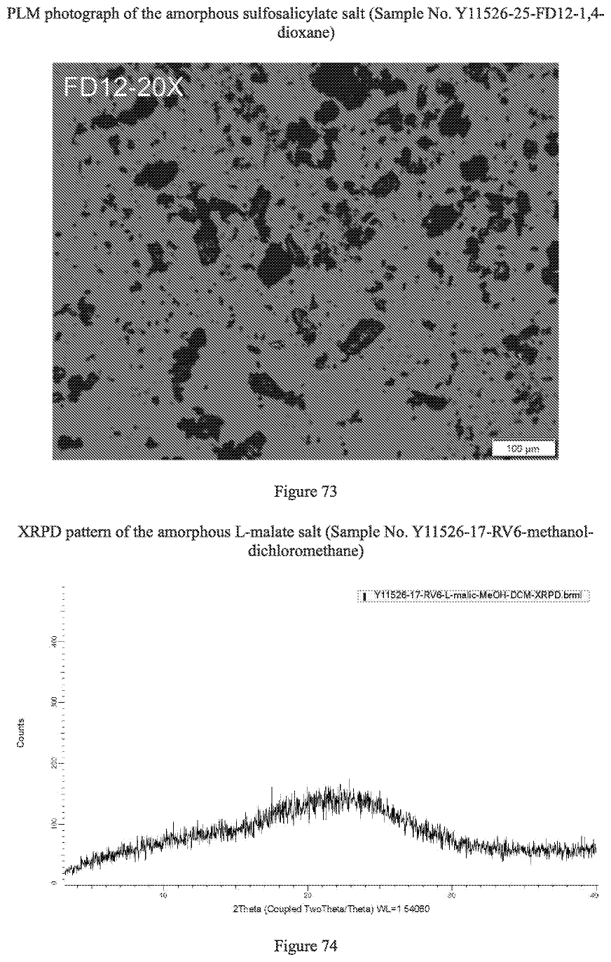

HPLC showed that the product had a purity of 99.8%. PLM showed that the product was an irregular sample (FIG. 73). XRPD showed that the product was amorphous (FIG. 69). DSC showed that the product had a glass-transition temperature of 86.3° C. (FIG. 70). TGA showed that the product had a weight loss of about 2.7% at 110° C. (FIG. 71). ¹H-NMR showed a base:acid stoichiometric ratio of about 1:1.5 in the product (FIG. 72), which was unreasonable.

The amorphous sulfosalicylate salt (Sample No. Y11526-25-FD12-1,4-dioxane) was chemically and physically stable, but unreasonable in terms of the base:acid stoichiometric ratio at the end of the preparation.

### Example 16: Amorphous L-Malate Salt (Sample No. Y1526-17-RV6-Methanol-Dichloromethane)

About 100 mg of the compound of formula (I) (with a purity of 99.9%) were weighed and placed in a 40 mL glass bottle together with 1 eq. of L-malic acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford a high-crystallinity material.

About 80 mg of the high-crystallinity material obtained were weighed and placed in a 40 mL glass bottle, and about 10 mL of methanol/dichloromethane (v:v=1:1) were added to obtain a clear solution. The clear solution was subjected to rapid volatilization to remove the solvent to afford the amorphous L-malate salt.

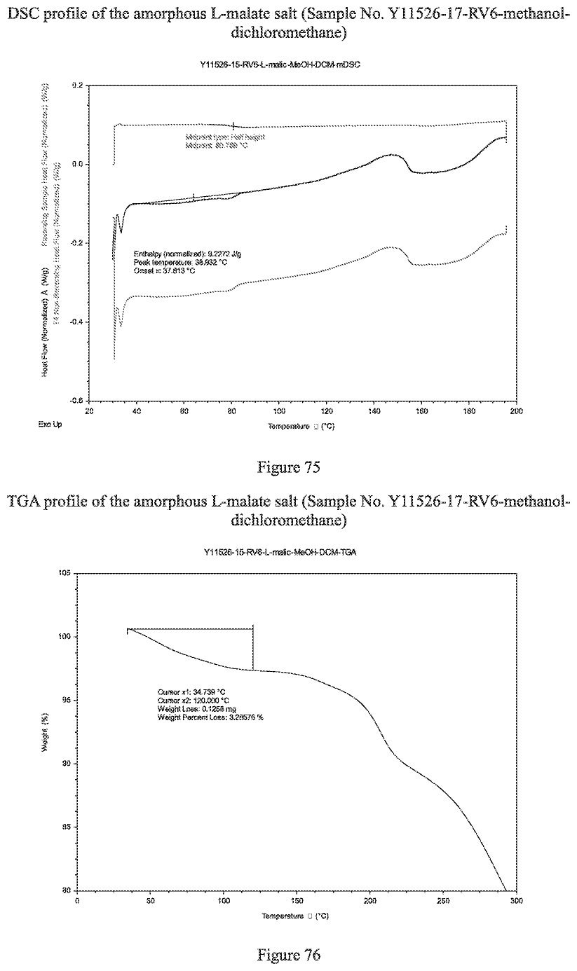

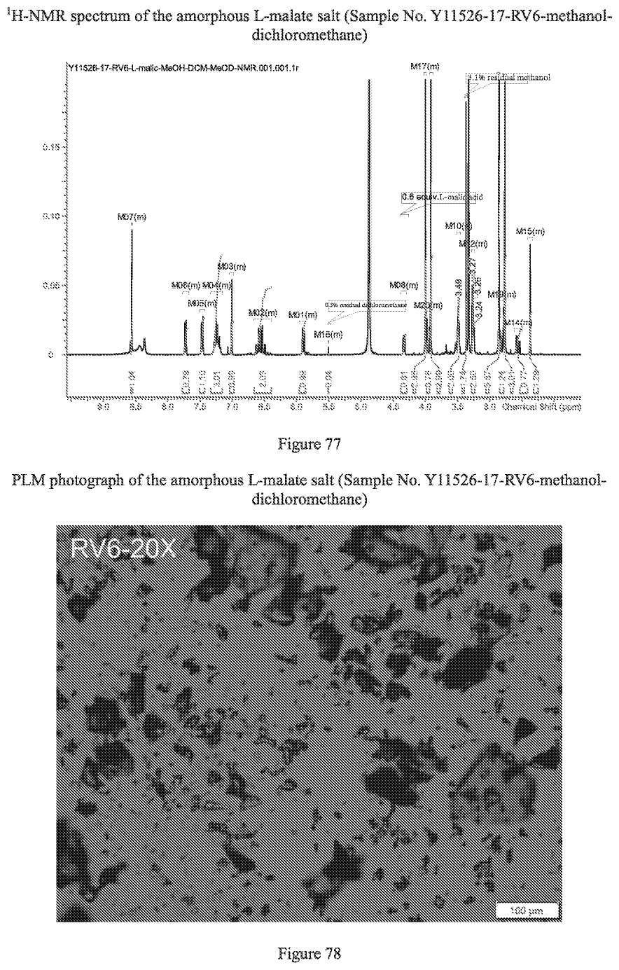

HPLC showed that the product had a purity of 91.1%, that is, the purity of the salt obtained at the end of the preparation was significantly reduced compared with the purity of the free base used to prepare the salt. PLM showed that the product was an irregular sample (FIG. 78). XRPD showed that the product was amorphous (FIG. 74). DSC showed that the product had a glass-transition temperature of 80.8° C. (FIG. 75). TGA showed that the product had a weight loss of about 3.3% at 120° C. (FIG. 76). ¹H-NMR showed a base:acid stoichiometric ratio of about 1:0.6 in the product (FIG. 77), which was unreasonable.

The amorphous L-malate salt (Sample No. Y11526-17-RV6-methanol-dichloromethane) was at least chemically unstable and unreasonable in terms of the base:acid stoichiometric ratio at the end of the preparation.

### Example 17: Amorphous 1 Eq. Citrate Salt (Sample No. Y11526-10-FD6-1,4-Dioxane)

About 100 mg of the compound of formula (I) (with a purity of 99.9%) were weighed and placed in a 40 mL glass bottle together with 1 eq. of citric acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford the amorphous 1 eq. citrate salt.

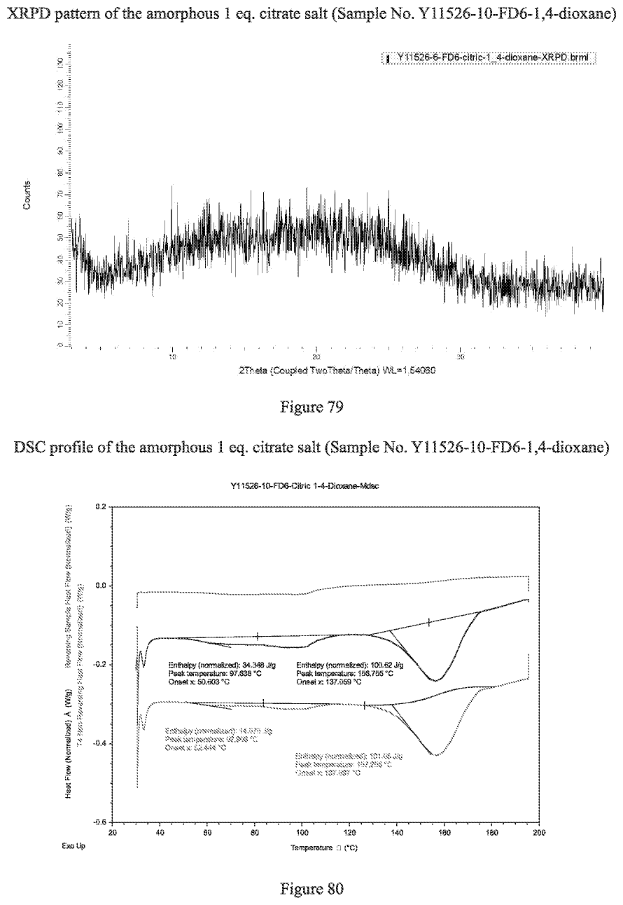

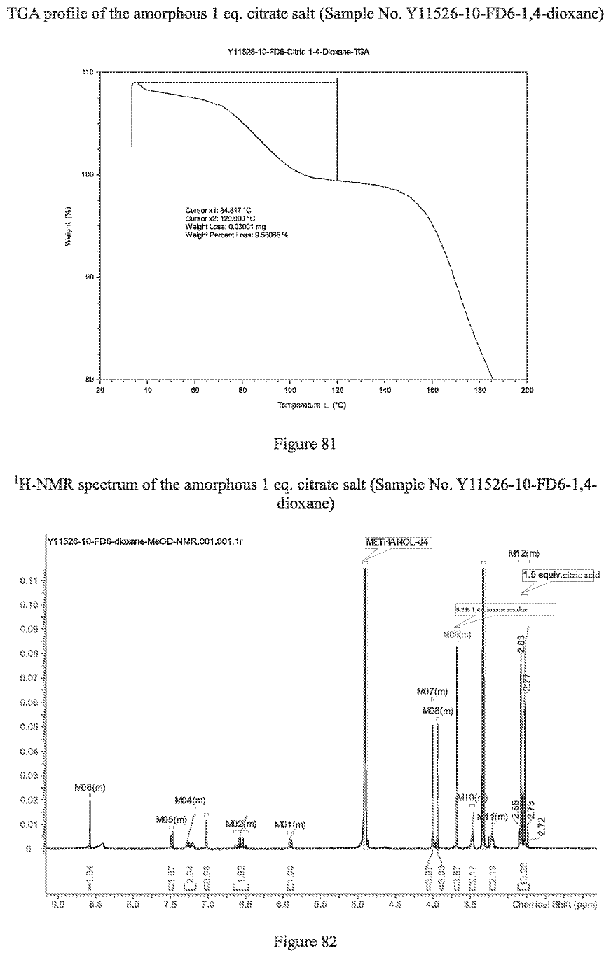

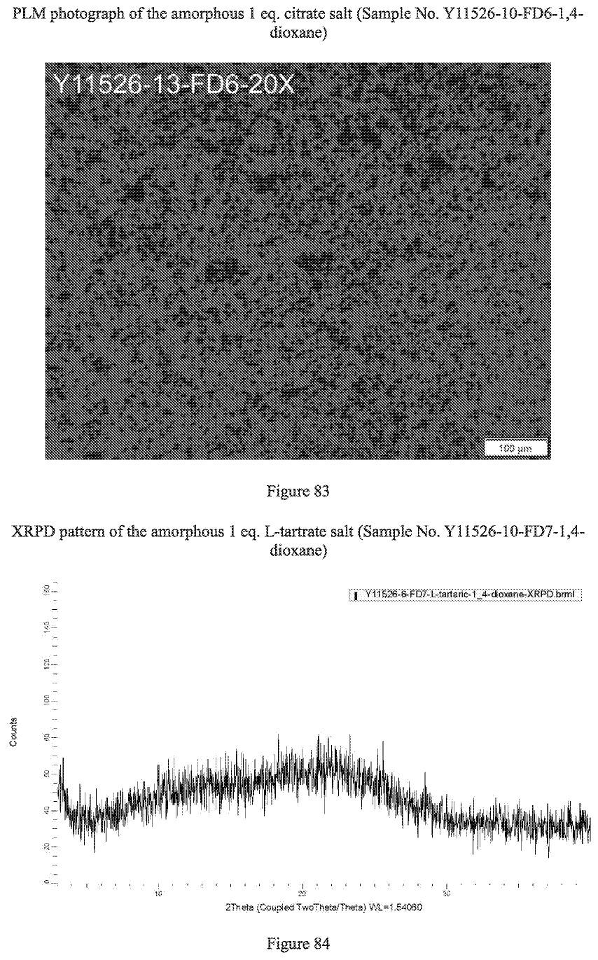

HPLC showed that the product had a purity of 94.4%, that is, the purity of the salt obtained at the end of the preparation was significantly reduced compared with the purity of the free base used to prepare the salt. PLM showed that the product was an irregular sample (FIG. 83). XRPD showed that the product was amorphous (FIG. 79). DSC showed that the product had no glass-transition temperature (FIG. 80). TGA showed that the product had a weight loss of about 9.6% at 120° C. (FIG. 81). ¹H-NMR showed a base:acid stoichiometric ratio of about 1:1 in the product (FIG. 82).

The amorphous 1 eq. citrate salt (Sample No. Y11526-10-FD6-1,4-dioxane) was reasonable in terms of the base:acid stoichiometric ratio, but at least chemically unstable at the end of the preparation.

### Example 18: Amorphous 1 Eq. L-Tartrate Salt (Sample No. Y11526-10-FD7-1,4-Dioxane)

About 100 mg of the compound of formula (I) (with a purity of 99.9%) were weighed and placed in a 40 mL glass bottle together with 1 eq. of L-tartaric acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford the amorphous 1 eq. L-tartrate.

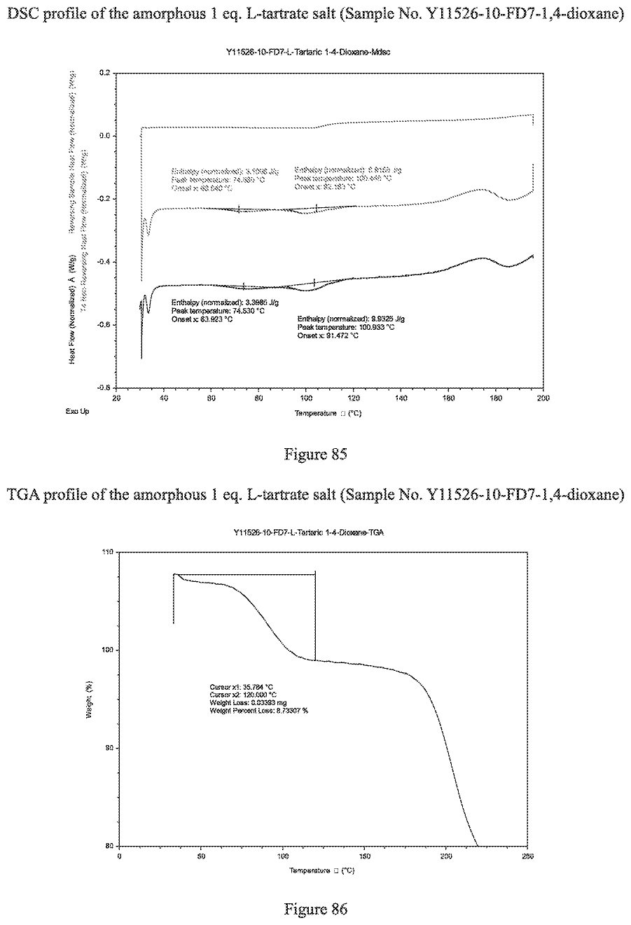

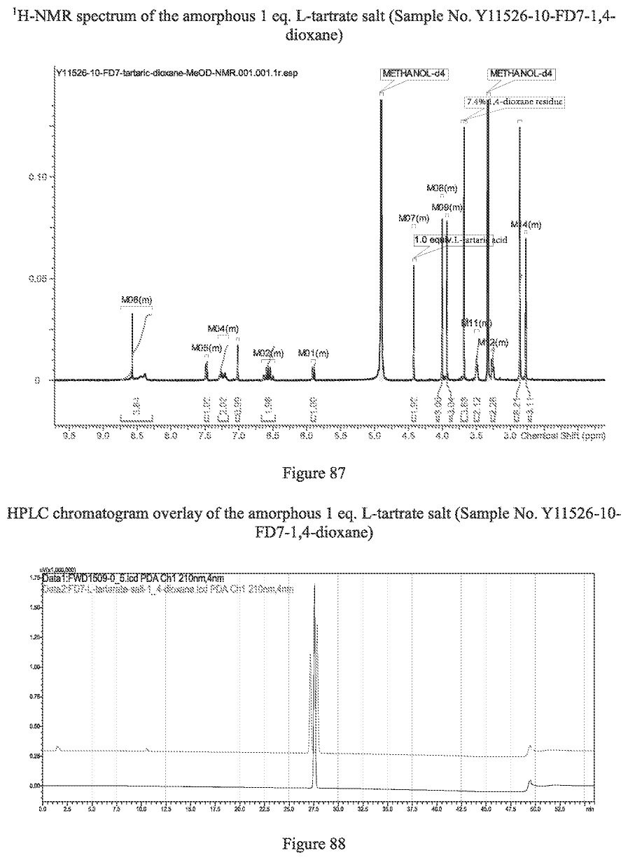

HPLC showed that the product was degraded (FIG. 88). XRPD showed that the product was amorphous (FIG. 84). DSC showed that the product had no glass-transition temperature (FIG. 85). TGA showed that the product had a weight loss of about 8.7% at 120° C. (FIG. 86). ¹H-NMR showed a base:acid stoichiometric ratio of about 1:1 in the product (FIG. 87).

The amorphous 1 eq. L-tartrate salt (Sample No. Y11526-10-FD7-1,4-dioxane) was reasonable in terms of the base:acid stoichiometric ratio, but at least chemically unstable at the end of the preparation.

It can be seen from Examples 1-18 that:

when the base:acid molar charge ratio was 1:1, sulfosalicylic acid and L-malic acid cannot enable the resulting salts to have a stoichiometric ratio of compound of formula (I):acid of 1:1;
when the base:acid molar charge ratio was 1:1, hydrochloric acid, methanesulfonic acid, benzenesulfonic acid, ethanesulfonic acid, maleic acid, hydrobromic acid, citric acid, and L-tartaric acid enable the resulting salts to have a stoichiometric ratio of compound of formula (I):acid of 1:1;

when the base:acid molar charge ratio was 1:2, nitric acid, sulfuric acid, and sulfosalicylic acid cannot enable the resulting salts to have a stoichiometric ratio of compound of formula (I):acid of 1:2; and

when the base:acid molar charge ratio was 1:2, hydrochloric acid, methanesulfonic acid, benzenesulfonic acid, and p-toluenesulfonic acid enable the resulting salts to have a stoichiometric ratio of compound of formula (I):acid of 1:2.

It can also be seen from Examples 1-18 that:

amorphous 1 eq. citrate salt, amorphous 1 eq. L-tartrate, amorphous nitrate salt and amorphous L-malate salt were chemically unstable at the end of the preparation;

amorphous 2 eq. p-tosylate salt and amorphous sulfate salt were physically unstable at the end of the preparation; and

amorphous 1 eq. hydrochloride salt, amorphous 2 eq. hydrochloride salt, amorphous 1 eq. mesylate salt, amorphous 2 eq. mesylate salt, amorphous 1 eq. besylate salt, amorphous 2 eq. besylate salt, amorphous 1 eq. esylate salt, amorphous 1 eq. maleate salt, crystalline 1 eq. hydrobromide salt, and amorphous sulfosalicylate salt were chemically and physically stable at the end of the preparation.

### Example 19: Solid Storage Stability Test

Each amorphous salt solid was weighed (two 2 mg samples were weighed for purity testing after solid storage stability study, and one 10 mg sample was weighed for XRPD testing after solid storage stability study) and placed in a glass vial. In a 25° C./60% RH solid storage stability test, the amorphous salt solid was placed in a 25° C./60% RH constant temperature and humidity chamber, and placed in the dark for 1 week (i.e. “solid/25° C./60% RH/open/1 week”), and in a 60° C. solid storage stability test, the amorphous salt solid was sealed in an oven at 60° C. and heated for 1 week in the dark (i.e. “solid/60° C./closed container/l week”). Then the sample was taken out for purity testing, crystal form detection and appearance observation, respectively.

Examples 1-10 were subjected to the above solid storage stability tests, and the results are shown in Table 1.

TABLE 1
Results of the solid storage stability test

| Examples | 1 | 2 | 3 | 4 | 5 |
|---|---|---|---|---|---|
| Sample | amorphous | amorphous | amorphous | amorphous | amorphous |
| | free base | 1 eq. | 2 eq. | 1 eq. | 2 eq. |
| | | hydrochloride salt | hydrochloride salt | mesylate salt | mesylate salt |
| Original | 99.9% | 99.9% | 99.9% | 99.9% | 99.9% |
| purity | | | | | |
| Original | amorphous | amorphous | amorphous | amorphous | amorphous |
| morphology | | | | | |

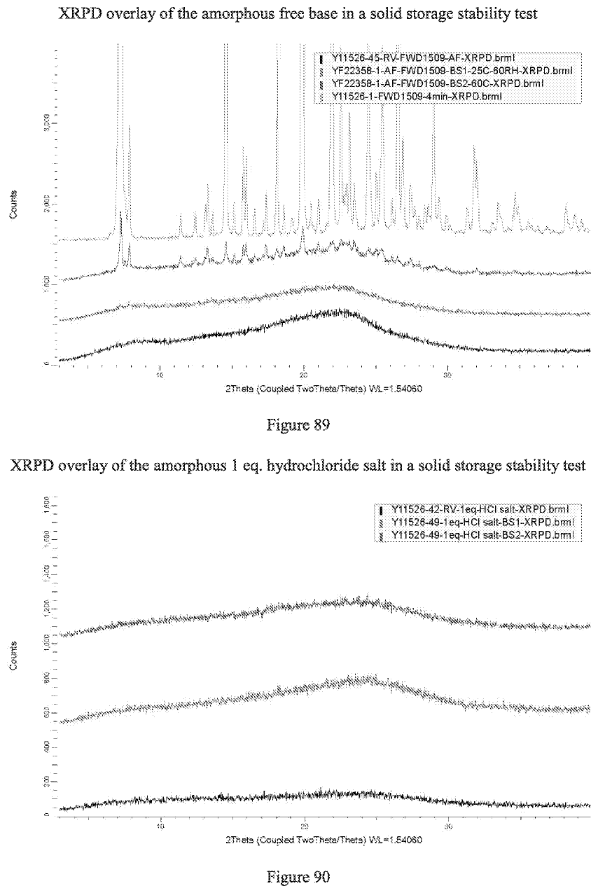

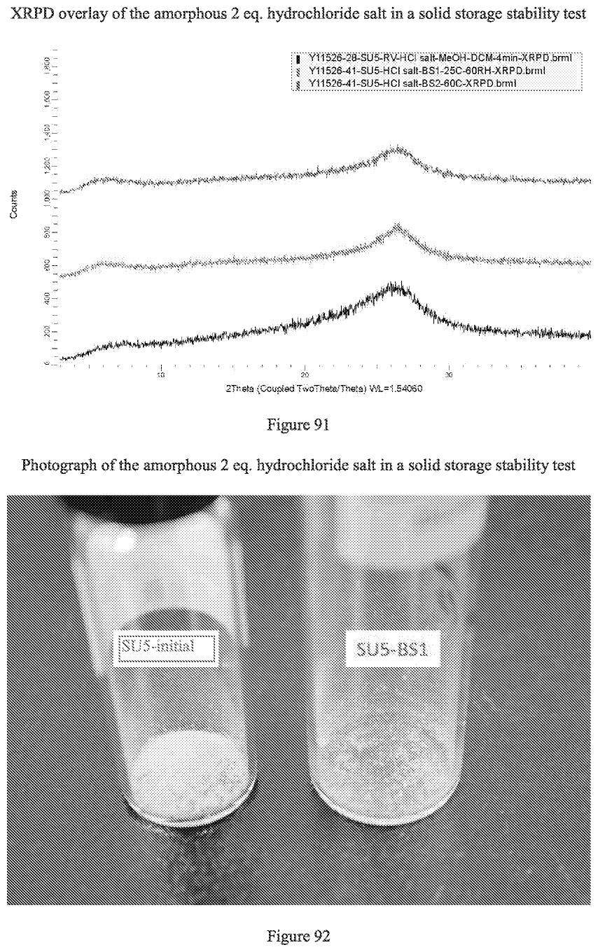

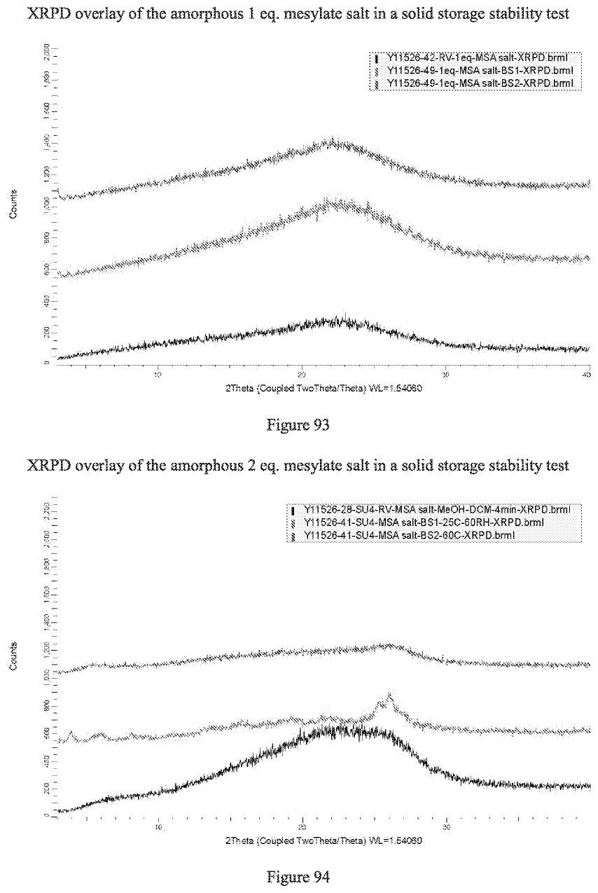

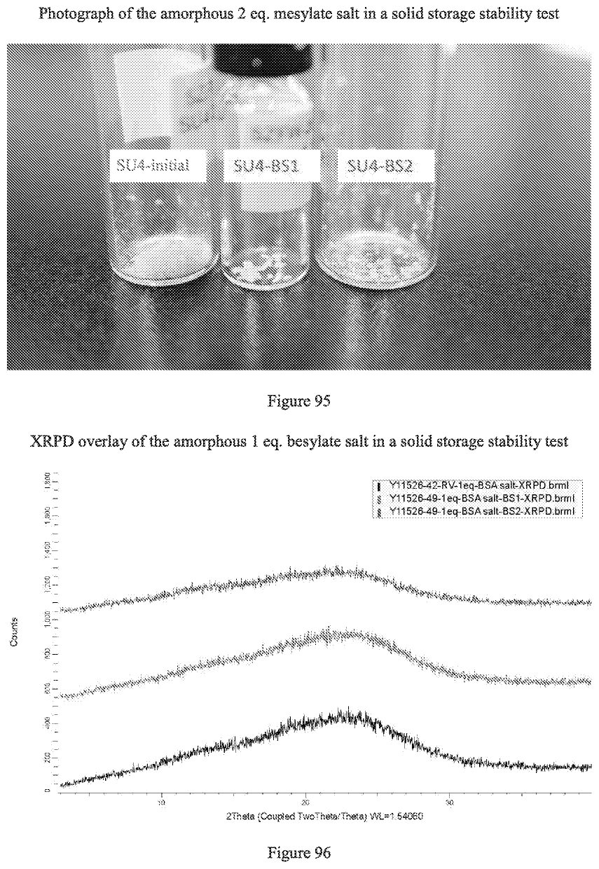

Test BS1: solid/25° C./60% RH/open/1 week

| | | | | | |
|---|---|---|---|---|---|
| Purity | 99.6% | 99.9% | 99.6% | 99.8% | 99.9% |
| Color | no color | no color | slight color | no color | no color |
| | change | change | change | change | change |
| Morphology | amorphous | amorphous | amorphous | amorphous | low |
| | (FIG. 89), | (FIG. 90), | (FIG. 91), | (FIG. 93), | crystallinity |
| | no | no | partial | no | (FIG. 94), |
| | agglomeration | agglomeration | agglomeration | agglomeration | agglomeration |
| | | | (FIG. 92) | | (FIG. 95) |

Test BS1: solid/60° C./closed container/1 week

| | | | | | |
|---|---|---|---|---|---|
| Purity | 99.9% | 98.2% | 99.6% | 99.9% | 99.8% |
| Color | no color | no color | no color | no color | no color |
| | change | change | change | change | change |
| Morphology | medium | amorphous | amorphous | amorphous | amorphous |
| | crystallinity | (FIG. 90), | (FIG. 91), | (FIG. 93), | (FIG. 94), |
| | (FIG. 89) | no | no | no | partial |
| | | agglomeration | agglomeration | agglomeration | agglomeration |
| | | | | | (FIG. 95) |

| Examples | 6 | 7 | 8 | 9 | 10 |
|---|---|---|---|---|---|
| Sample | amorphous | amorphous | amorphous | amorphous | crystalline |
| | 1 eq. | 2 eq. | 1 eq. | 1 eq. | 1 eq. |
| | besylate salt | besylate salt | esylate salt | maleate salt | hydrobromide salt |
| Original | 99.7% | 99.6% | 99.8% | 99.8% | 99.9% |
| purity | | | | | |
| Original | amorphous | amorphous | amorphous | amorphous | crystalline |
| morphology | | | | | |

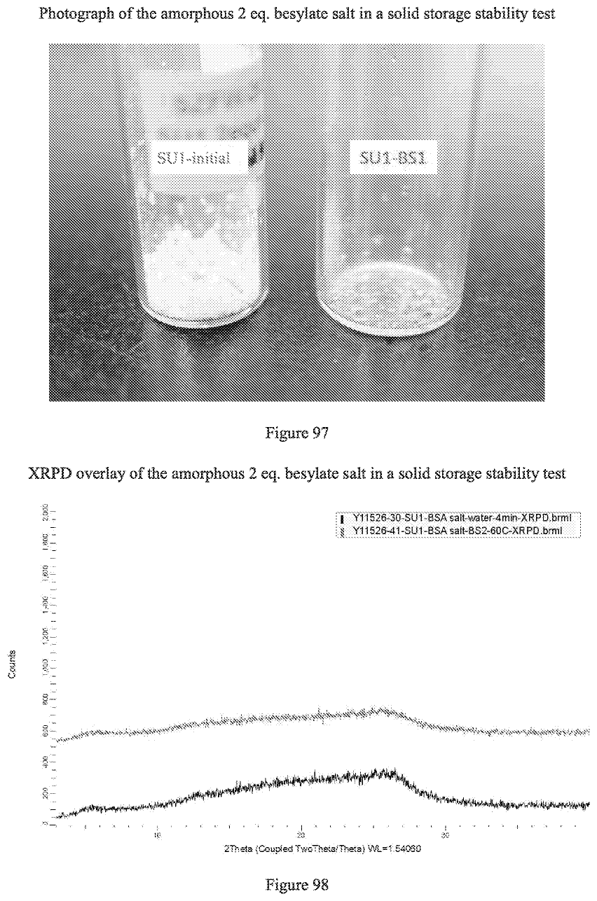

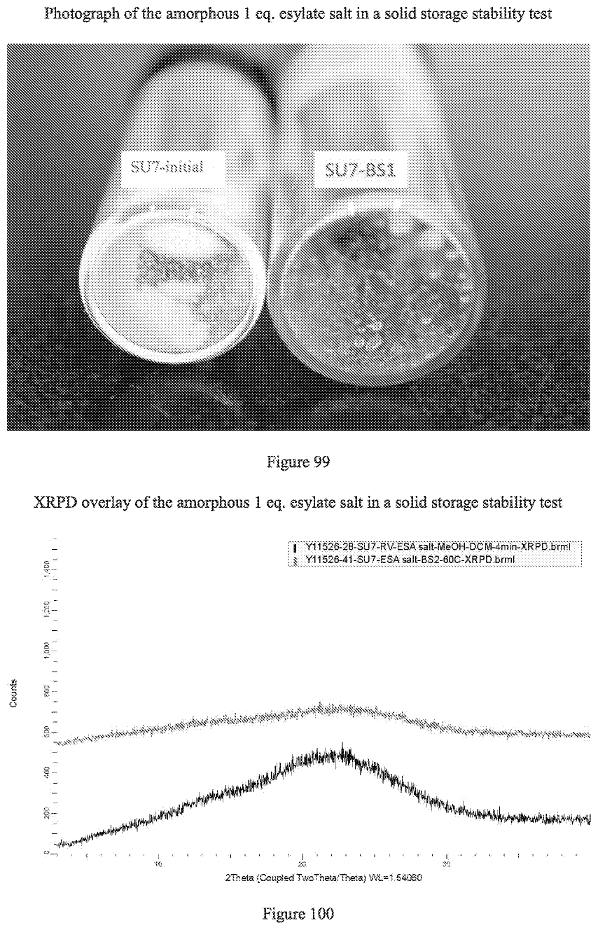

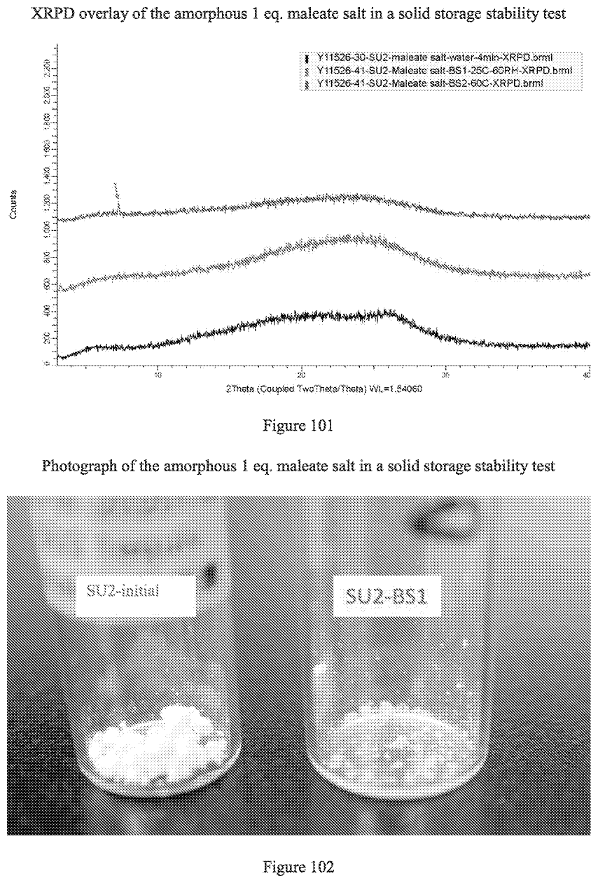

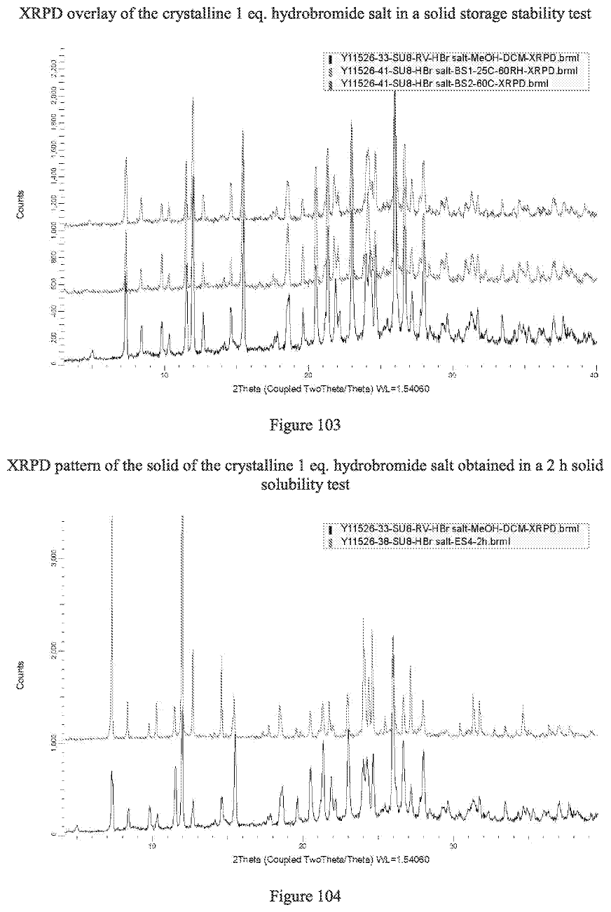

Test BS1: solid/25° C./60% RH/open/1 week

| | | | | | |
|---|---|---|---|---|---|
| Purity | 98.2% | 99.6% | 99.9% | 99.7% | 99.9% |
| Color | no color- | slight color | no color | slight color- | no color |
| | change | change | change | change | change |
| Morphology | amorphous | caking and | caking and | amorphous | no crystal |
| | (FIG. 96), | fusion to a | fusion to a | (FIG. 101), | form change |
| | no | glassy state | glassy state | agglomeration | (Figure 103) |
| | agglomeration | (FIG. 97) | (FIG. 99) | (FIG. 102) | |

Test BS1: solid/60° C./closed container/1 week

| | | | | | |
|---|---|---|---|---|---|
| Purity | 99.6% | 99.4% | 99.8% | 98.2% | 99.9% |
| Color | no color | no color | no color | no color | no color |
| | change | change | change | change | change |
| Morphology | amorphous | amorphous | amorphous | low | no crystal |
| | (FIG. 96), | (FIG. 98), | (FIG. 100), | crystallinity | form change |
| | no | no | no | (FIG. 10.1) | (Figure 103) |
| | agglomeration | agglomeration | agglomeration | | |

The amorphous free base showed good physical and chemical stability under the storage stability test conditions of “solid/25° C./60% RH/open/1 week”, and transformed from amorphous to a moderate crystallinity under the storage stability test conditions of “solid/60° C./closed container/1 week”.

The amorphous 1 eq. hydrochloride salt showed good physical and chemical stability under the storage stability test conditions of “solid/25° C./60% RH/open/1 week”, and had a decrease in purity (about 2%) under the storage stability test conditions of “solid/60° C./closed container/1 week”.

The amorphous 2 eq. hydrochloride salt had slight color change and showed a certain hygroscopicity (partial agglomeration) under the storage stability test conditions of “solid/25° C./60% RH/open/1 week”, and showed good physical and chemical stability under the storage stability test conditions of “solid/60° C./closed container/1 week”.

The amorphous 1 eq. mesylate salt showed good physical and chemical stability under the storage stability test conditions of both “solid/25° C./60% RH/open/1 week” and “solid/60° C./closed container/1 week”.

The amorphous 2 eq. mesylate salt transformed from amorphous to a low crystallinity and showed a certain hygroscopicity (agglomeration) under the storage stability test conditions of “solid/25° C./60% RH/open/1 week”, and showed a certain hygroscopicity (partial agglomeration) under the storage stability test conditions of “solid/60° C./closed container/1 week”.

The amorphous 1 eq. besylate salt had a decrease in purity (about 2%) under the storage stability test conditions of “solid/25° C./60% RH/open/1 week”, and showed good physical and chemical stability under the storage stability test conditions of “solid/60° C./closed container/1 week”.

The amorphous 2 eq. besylate salt had slight color change and showed significant hygroscopicity (caking and fusion to a glassy state) under the storage stability test conditions of “solid/25° C./60% RH/open/1 week”, and showed good physical and chemical stability under the storage stability test conditions of “solid/60° C./closed container/l week”.

The amorphous 1 eq. esylate salt showed significant hygroscopicity (caking and fusion to a glassy state) under the storage stability test conditions of “solid/25° C./60% RH/open/1 week”, and showed good physical and chemical stability under the storage stability test conditions of “solid/60° C./closed container/1 week”.

The amorphous 1 eq. maleate salt had slight color change and showed a certain hygroscopicity (agglomeration) under the storage stability test conditions of “solid/25° C./60% RH/open/l week”, and had a decrease in purity (about 2%) and transformed from amorphous to a low crystallinity under the storage stability test conditions of “solid/60° C./closed container/1 week”.

The crystalline 1 eq. hydrobromide salt showed good physical and chemical stability under the storage stability test conditions of both “solid/25° C./60% RH/open/1 week” and “solid/60° C./closed container/I week”.

It can be seen from Example 19 that:

The amorphous 1 eq. besylate salt, amorphous 1 eq. hydrochloride salt and amorphous 1 eq. maleate salt were chemically unstable under the storage conditions of “solid/25° C./60% RH/open/1 week” and/or “solid/60° C./closed container/1 week”;

the amorphous 2 eq. hydrochloride salt, amorphous 2 eq. mesylate salt, amorphous 2 eq. besylate salt, amorphous 1 eq. esylate salt, and amorphous 1 eq. maleate salt were physically unstable under the storage conditions of “solid/25° C./60% RH/open/1 week” and/or “solid/60° C./closed container/I week”; and

the amorphous 1 eq. mesylate salt and crystalline 1 eq. hydrobromide salt were chemically and physically stable under the storage conditions of “solid/25° C./60% RH/open/1 week” and “solid/60° C./closed container/1 week”.

### Example 20: Solid Solubility Test

samples were weighed for each amorphous salt solid (equivalent to 20 mg of free base), and 10 mL of the following solvents were respectively added: 0.1N HCl solution (pH 1.0), 50 mM phosphate buffer (pH 4.5), FeSSIF-V1 (pH 5.0), FaSSIF-V1 (pH 6.5) and SGF (pH 2.0), and stirred at 37° C. for 2 h. The suspension was then centrifuged at 37° C., the supernatant was measured for solubility by HPLC, and the solid fraction was measured for XRPD. The target solubility was at least 2 mg (based on free base)/mL.

Examples 1-10 were subjected to the above 2 h solid solubility test, and the results are shown in Table 2. Similarly, Examples 1 and 4 were subjected to a 24 h solid solubility test, and the results are shown in Table 3.

TABLE 2
Results of the 2 h solid solubility test
Examples

| | | |
|---|---|---|
| 1 | 2 | 3 |

Sample

| amorphous | | amorphous 1 eq. | | amorphous 2 eq. |
|---|---|---|---|---|
| free base | | hydrochloride salt | | hydrochloride salt |
| Solubility | XRPD | Solubility | XRPD | Solubility |
| measured | measured | measured | measured | measured |
| at 2 h (pH) | at 2 h | at 2 h (pH) | at 2 h | at 2 h (pH) |

Test ES1: 0.1N HCl solution (pH 1.0)

| | | | | |
|---|---|---|---|---|
| >2 mg/mL | clear solution | >2 mg/mL | clear solution | >2 mg/mL |
| (0.8) | not performed | (0.7) | not performed | (0.71 |

Test ES2: 50 mM phosphate buffer (pH 4.5)

| | | | | |
|---|---|---|---|---|
| >2 mg/mL | clear solution | >2 mg/mL | clear solution | >2 mg/mL |
| (4.6) | not performed | (4.5) | not performed | (4.2) |

Test ES3: FeSSIF-V1 (pH 5.0)

| | | | | |
|---|---|---|---|---|
| >2 mg/mL | clear solution | >2 mg/mL | clear solution | >2 mg/mL |
| (4.9) | not performed | (4.8) | not performed | (4.8) |

Test ES1: FaSSIF-V1 (pH 6.5)

| | | | | |
|---|---|---|---|---|
| >2 mg/mL | clear solution | >2 mg/mL | clear solution | >2 mg/mL |
| (6.8) | not performed | (6.5) | not performed | (6.1) |

Test ES5: SGF (pH 2.0)

| | | | | |
|---|---|---|---|---|
| >2 mg/mL | clear solution | >2 mg/mL | clear solution | >2 mg/mL |
| (2.4) | not performed | (2.0) | not performed | (1.8) |

Examples

| | | |
|---|---|---|
| 3 | 4 | 5 |

Sample

| amorphous 2 eq. | amorphous 1 eq. | | amorphous 2 eq. | |
|---|---|---|---|---|
| hydrochloride salt | mesylate salt | | mesylate salt | |
| XRPD | Solubility | XRPD | Solubility | XRPD |
| measured | measured | measured | measured | measured |
| at 2 h | at 2 h (pH) | at 2 h | at 2 h (pH) | at 2 h |
| Test ES1: 0.1N HCl solution (pH 1.0) | | | | |
| clear solution | >2 mg/mL | clear solution | >2 mg/mL | clear solution |
| not performed | (0.7) | not performed | (0.7) | not performed |
| Test ES2: 50 mM phosphate buffer (pH 4.5) | | | | |
| clear solution | >2 mg/mL | clear solution | >2 mg/mL | clear solution |
| not performed | (4.5) | not performed | (4.2) | not performed |
| Test ES3: FeSSIF-V1 (pH 5.0) | | | | |
| clear solution | >2 mg/mL | clear solution | >2 mg/mL | clear solution |
| not performed | (4.8) | not performed | (4.8) | not performed |
| Test ES1: FaSSIF-V1 (pH 6.5) | | | | |
| clear solution | >2 mg/mL | clear solution | >2 mg/mL | clear solution |
| not performed | (6.5) | not performed | (6.2) | not performed |
| Test ES5: SGF (pH 2.0) | | | | |
| clear solution | >2 mg/mL | clear solution | >2 mg/mL | clear solution |
| not performed | (2.0) | not performed | (1.8) | not performed |

Examples

| | | |
|---|---|---|
| 6 | 7 | 8 |

Sample

| amorphous 1 eq, | | amorphous 2 eq. | | amorphous 1 eq. |
|---|---|---|---|---|
| besylate salt | | besylate salt | | esylate salt |
| Solubility | XRPD | Solubility | XRPD | Solubility |
| measured | measured | measured | measured | measured |
| at 2 h (pH) | at 2 h | at 2 h (pH) | at 2 h | at 2 h (pH) |

Test ES1: 0.1N HC1 solution (pH 1.0)

| | | | | |
|---|---|---|---|---|
| >2 mg/mL | clear solution | 1.8 mg/mL | nearly | >2 mg/mL |
| (0.7) | not performed | (0.8) | clear solution | (0.8) |
| | | | a small amount | |
| | | | of floc | |
| | | | not performed | |

Test ES2: 50 mM phosphate buffer (pH 4.5)

| | | | | |
|---|---|---|---|---|
| >2 mg/mL | clear solution | >2 mg/mL | clear solution | >2 mg/mL |
| (4.5) | not performed | (4.2) | not performed | (4.4) |

Test ES3: FeSSIF-V1 (pH 5.0)

| | | | | |
|---|---|---|---|---|
| >2 mg/mL | clear solution | >2 mg/mL | clear solution | >2 mg/mL |
| (4.8) | not perforated | L(4.8) | not performed | (4.8) |

Test ES4: FaSSIF-V1 (pH 6.5)

| | | | | |
|---|---|---|---|---|
| >2 mg/mL | clear solution | >2 mg/mL | clear solution | >2 mg/mL |
| (6.5) | not performed | (6.1) | not performed | (6.4) |

Test ES5: SGF (pH 2.0)

| | | | | |
|---|---|---|---|---|
| >2 mg/mL | clear solution | >2 mg/mL | clear solution | >2 mg/mL |
| (2.1) | not performed | (1.7) | not performed | (1.9) |

Examples

| | | |
|---|---|---|
| 8 | 9 | 10 |

Sample

| amorphous 1 eq. | amorphous 1 eq. | | crystalline 1 eq. | |
|---|---|---|---|---|
| esylate salt | maleate salt | | hydrobromide salt | |
| XRPD | Solubility | XRPD | Solubility | XRPD |
| measured | measured | measured | measured | measured |
| at 2 h | at 2 h (pH) | at 2 h | al 2 h (pH) | at 2 h |
| Test ES1: 0.1N HC1 solution (pH 1.0) | | | | |
| clear solution | >2 mg/mL | clear solution | >2 mg/mL | clear solution |
| not performed | (0.8) | not performed | (0.8) | not performed |
| Test ES2: 50 mM phosphate buffer (pH 4.5) | | | | |
| clear solution | >2 mg/mL | clear solution | >2 mg/mL | clear solution |
| not performed | (4.3) | not performed | (4.4) | not performed |
| Test ES3: FeSSIF-V1 (pH 5.0) | | | | |
| clear solution | >2 mg/mL | clear solution | >2 mg/mL | clear solution |
| not performed | (4.8) | not performed | (4.8) | not performed |
| Test ES4: FaSSIF-V1 (pH 6.5) | | | | |
| clear solution | >2 mg/mL | clear solution | 0.7 mg/mL | hydrobromide |
| not performed | (6.1) | not performed | (6.4) | salt FIG. 104 |
| Test ES5: SGF (pH 2.0) | | | | |
| clear solution | >2 mg/mL | clear solution | >2 mg/mL | clear solution |
| not performed | (2.0) | not performed | (1.9) | not perforated |

TABLE 3
Results of the 24 h solid, solubility test

| Examples | | | |
|---|---|---|---|
| 1 | | 4 | |
| Sample | | | |
| amorphous free base | | amorphous 1 eq. mesylate salt | |
| Solubility | XRPD | Solubility | XRPD |
| measured at | measured at | measured at | measured at |
| 24 h (pH) | 2.4 h | 24 h (pH) | 2.4 h |

Test ES3: FeSSIF-VI (pH 5.0)

| | | | |
|---|---|---|---|
| >2 mg/mL | clear solution | >2 mg/mL | clear solution |
| (5.0) | not | (5.0) | not |
| | performed | | performed |

Test ES4: FaSSIF-VI (pH 6.5)

| | | | |
|---|---|---|---|
| >2 mg/mL | clear solution | >2 mg/mL | clear solution |
| (6.8) | not | (6.6) | not |
| | performed | | performed |

In the 2 h solid solubility test, the amorphous 2 eq. besylate salt did not achieve a solubility of 2 mg/mL in the 0.1N HCl solution (pH 1.0), the crystalline 1 eq. hydrobromide salt did not achieve a solubility of 2 mg/mL in FaSSIF-V1 (pH 6.5), and the amorphous 1 eq. hydrochloride salt, amorphous 2 eq. hydrochloride salt, amorphous 1 eq. mesylate salt, amorphous 2 eq. mesylate salt, amorphous 1 eq. besylate salt, amorphous 1 eq. esylate salt, and amorphous 1 eq. maleate salt had a solubility greater than 2 mg/mL in all solvents tested. Additionally, the amorphous free base and the amorphous 1 eq. mesylate salt had a solubility greater than 2 mg/mL in FeSSIF-V1 (pH 5.0) and FaSSIF-V1 (pH 6.5) in the 24 h solid solubility test.

It can be seen from Examples 1-20 that the amorphous 1 eq. mesylate salt had a reasonable salt-forming equivalent ratio, better chemical stability, better physical stability and better solubility simultaneously.

Although the invention has been described in detail by the detailed description and examples for clarity, these description and examples should not be construed as limiting the scope of the present invention.

1. A salt formed from a compound of formula (I) with an acid: 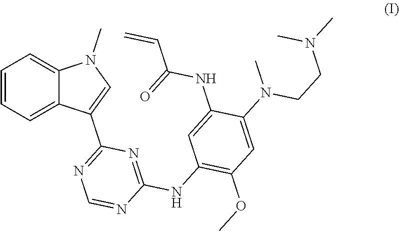 [SMILES: C=CC(=O)Nc1cc(Nc2ncnc(-c3cn(C)c4ccccc34)n2)c(OC)cc1N(C)CCN(C)C]
wherein the acid is selected from hydrochloric acid, methanesulfonic acid, benzenesulfonic acid, ethanesulfonic acid, maleic acid, hydrobromic acid, citric acid, L-tartaric acid, and p-toluenesulfonic acid.

2. The salt according to claim 1, wherein the acid is selected from hydrochloric acid, methanesulfonic acid, and maleic acid.

3. The salt according to claim 1, wherein the equivalent ratio of the compound of formula (I) to the acid is 1:1 or 1:2.

4. The salt according to claim 3, wherein the acid is methanesulfonic acid.

5. The salt according to claim 4, wherein the equivalent ratio of the compound of formula (I) to the acid is 1:1.

6. A pharmaceutical composition comprising the salt according to claim 1 and a pharmaceutically acceptable carrier.

7. A method of treating non-small cell lung cancer (NSCLC) with EGFR exon 20 insertion mutation, comprising administering the salt according to claim 1 to a patient.

8. (canceled)

9. A method of treating non-small cell lung cancer (NSCLC) with EGFR exon 20 insertion mutation, comprising administering the pharmaceutical composition according to claim 6 to a patient.
